(19) United States
(12) Patent Application Publication
(10) Pub. No.: US 2003/0085388 A1
(43) Pub. Date: May 8, 2003

(54) Novel nonlinear optical compounds and polymers

(76) Inventor: Luping Yu, Chicago, IL (US)

Correspondence Address: BRINKS HOFER GILSON & LIONE, P.O. BOX 10395, CHICAGO, IL, 60610, US

(21) Appl. No.: 10/196,734

(22) Filed: Jul. 15, 2002

Related U.S. Application Data
(60) Provisional application No. 60/305,374, filed on Jul. 13, 2001.

(51) Int. Cl. C 07D 4 9/14; G02B 5/02

(52) U.S. Cl. 252/582; 549/59; 544/300; 548/440

(57) ABSTRACT

Novel compositions and synthetic methods for forming nonlinear optic polymers, which may be incorporated into multiple light-based devices, are disclosed. These compositions include chromophoric monomer units that incorporate nonlinear optic chromophores, linking monomers that may be used to link chromophoric monomers, and polymers made from chromophoric monomers or chromophoric monomers in combination with linking monomers. The polymers can exhibit high thermal stability, which is believed to arise from their covalently bonded chromophore structures. In addition to their covalently bonded chromophore structures, in one aspect, nonlinear optic polymers are disclosed that may be crosslinked to further increase the thermal and dipole stability of the polymers.

### REFERENCE TO RELATED APPLICATIONS

This application claims the benefit of U.S. Provisional Application No. 60/305, 374, filed Jul. 13, 2001, entitled “Novel Nonlinear Optical Polyesterimides Containing Chromophores with Large Hyperpolarizabilities,” the contents of which are incorporated herein by reference in its entirety.

### BACKGROUND

A limitation in the rate at which information may be transmitted over systems that rely on combinations of fiber optic transmission with digital switching is emerging. A desirable solution is to use broadband fiber optics, which allow for significantly more efficient information processing and transmission, in combination with light-based optical switching. In this fashion, the digital domain bottlenecks that are currently required to process or switch the light-based information may be reduced or eliminated. In developing the fiber optic transmission systems of the future, nonlinear optical (NLO) devices, such as high frequency modulators, waveguides, and directional couplers, are expected to play a key role.

Nonlinear optic materials are capable of varying their refractive index in the presence of an applied voltage or field. For example, electro-optical NLO devices can change their refractive index in response to application of an electric field. A more complete discussion of nonlinear optical materials may be found in D. S. Chemla and J. Zyss, Nonlinear optical properties of organic molecules and crystals, Academic Press, 1987.

For preparing NLO devices, nonlinear optical materials exhibiting large electro-optic (EO) coefficients are vital. Thus, materials that exhibit highly nonlinear optical characteristics of doubling the frequency of incident light are of great interest. Due to their high dielectric constants, however, prior inorganic NLO electro-optic (EO) materials are limited in the highest frequency they can achieve.

Due to the deficiencies of inorganic NLO materials, manifested in the mismatch of refractive index (n) and dielectric constant (d), polymers that exhibit large EO values due to their conjugated π-electron chromophores are expected to find extensive use in opto-electronic applications. While polymers functionalized with NLO chromophores have been studied, significant deficiencies remain.

Larger nonlinear optical (NLO) responses and a higher thermal stability in dipole orientation are two key requirements in the development of second order polymeric NLO materials for practical applications. Despite past progress in the synthesis of NLO chromophores that exhibit large EO values, conventional methods fail to provide viable means to anchor these chromophores to polymer backbones possessing high glass transition temperatures. The difficulty arises from the fact that most of these NLO chromophores are very sensitive towards chemical manipulation and are rarely compatible with many polymerization conditions. For example, the amine monomers used in polyimide synthesis routinely cause decomposition of the NLO chromophores possessing the top EO values.

Thus, many disadvantages exist in conventional polymeric nonlinear optical materials. One is a lack of polymer backbone structures to which a wide variety of chromophoric side-chains may be attached. Additionally, there is a lack of polymer backbones that can carry a high density of chromophoric side-chains, as required for large nonlinearity effects (EO). Another disadvantage is a lack of polymer structures with chromophoric side-chains that have a high glass transition temperature T_{g}. A high T_{g} is desirable because it provides for high thermal stability during processing. Other disadvantages of known NLO polymers include the lack of polymeric backbones having uniform side-chain functionalization and a lack of polymer uniformity in general, which interfere with device fabrication.

Another deficiency of current NLO polymers is often referred to as optical loss. Optical loss may arise from multiple sources, including, scattering losses due to defects and impurities in the polymer films, absorption losses due to photoinduced electronic transitions, and absorption losses due to vibrational transition involving C—H bonds.

As can be seen from the above description, there is an ongoing need for new NLO polymeric materials and their methods of synthesis. The polymeric materials of the present invention overcome at least one or more of the disadvantages associated with conventional NLO polymers and methods of synthesis.

### SUMMARY

In one embodiment, the invention provides compounds for forming NLO materials.

In another embodiment, the invention provides compounds for forming NLO chromophoric monomers.

In another embodiment, the invention provides NLO polymers comprising chromophoric monomers.

In another embodiment, the invention provides NLO polymers comprising chromophoric monomers and linking monomers.

In another embodiment, the invention provides NLO polymers comprising chromophoric monomers and crosslinkable linking monomers.

In another embodiment, the invention provides methods of making NLO polymers.

In another embodiment, the invention provides electro-optical devices comprising NLO polymers.

The scope of the present invention is defined solely by the appended claims, and is not affected to any degree by the statements within this summary.

### BRIEF DESCRIPTION OF THE DRAWINGS

FIG. 1 is an illustrative synthetic approach embodying features of the current invention for NLO monomers 11a-c, where the sensitive NLO chromophores were prepared in the last step to reduce decomposition.

FIG. 2 is an illustrative synthetic approach embodying features of the current invention for NLO monomers 20a-c, where the sensitive NLO chromophores were prepared in the last step to reduce decomposition.

FIG. 3 is an illustrative synthetic approach embodying features of the current invention for a linking monomer.

FIG. 4 is an illustrative synthetic approach embodying features of the current invention for NLO polymers having polyester imide functionality.

FIG. 5 is an illustrative synthetic approach embodying features of the current invention for crosslinkable linking monomers, 34 and 34-1, 34-2 and a thermally crosslinkable NLO polymer, 41a-d.

FIG. 6 is an illustrative synthetic approach embodying features of the current invention for dihydroxyl NLO monomers, where the sensitive NLO chromophores were prepared in the last step to reduce decomposition.

FIG. 7 is an illustrative synthetic approach embodying features of the current invention for monohydroxyl NLO monomers, where the sensitive NLO chromophores were prepared in the last step to reduce decomposition.

FIG. 8 is an illustrative synthetic approach embodying features of the current invention for dihalogen NLO monomers, where the sensitive NLO chromophores were prepared in the last step to reduce decomposition.

FIG. 9 is a plot showing the absorption change of a NLO polymer embodying features of the current invention before and after poling.

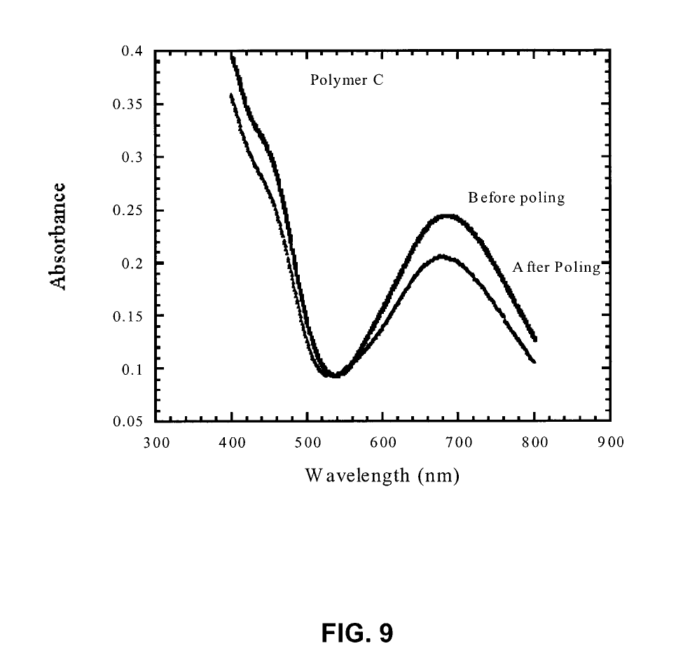

FIG. 10 is a plot showing the temporal stability of three NLO polymers embodying features of the current invention.

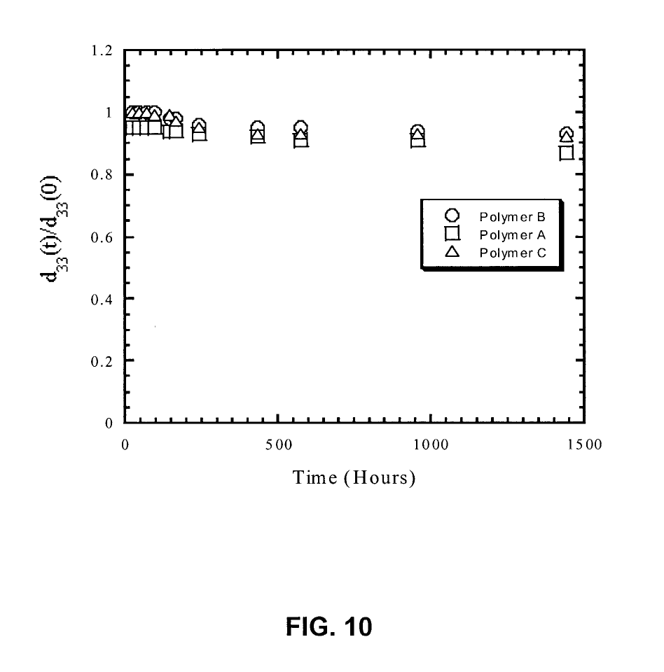

FIG. 11 is a table listing some physical properties, including glass transition temperature (T_{g}) and decomposition temperature (T_{d}), of NLO polymers embodying features of the current invention.

### DETAILED DESCRIPTION

### Definitions

Single Bond

A single bond exists when two atoms each share an electron with the other atom to form a bond. The existence of shared bonding electrons provides an aggregate with sufficient stability to consider it as an independent molecular species. Examples include covalent bonds between carbon atoms, such as those found in alkanes; covalent bonds between carbon and hetero-atoms (including nitrogen and oxygen), as found in alcohols and amide groups.

In the current specification and appended claims, single bonds are represented as solid or dashed lines. They are generally represented as dashed lines when depicting single bonding between interchangeable groups. For example, in the structure CH₃—CH₂—CH₂— —R, wherein R can be —OH or —Cl, it is understood that the actual structure can be CH₃—CH₂—CH₂—OH or CH₃—CH₂—CH₂—Cl.

In many instances, a dashed bond ends in braces containing the moiety to which the group is bonded. Thus, a structure of the type R-{To X} means that group R is bonded to group X. It should be understood that {To X} includes the circumstances when R is not directly bonded to X, such as when one or more additional groups or spacer moieties are bonded between R and X. For example, in the structure R-{To X}, it is understood that the actual arrangement could be R—X, R-A-X, or R-A-B-X, wherein A and B are other groups or spacer moieties.

Polymer

Polymers are composed of many smaller, covalently bonded units, known as monomer units. Multiple monomer units are covalently attached to form the backbone of a polymer. In one aspect, a polymer may include a single repeating monomer unit. In another aspect, polymers are made from at least two different monomer units and may be referred to as copolymers. In yet another aspect, a polymer may include larger repeating units where each repeating unit includes multiple monomer units. These types of polymers are often referred to as block copolymers. In accordance with the present invention, various monomers and monomer units may be combined to form a plethora of NLO polymers, copolymers, and block copolymers.

Polymerizing or copolymerizing describes the process by which multiple monomers (i.e. chemical compounds) are reacted to form covalently linked monomer units that form polymers or copolymers, respectively. A discussion of polymers, monomer units, and the monomers from which they are made may be found in Stevens, Polymer Chemistry: An Introduction, 3^{rd} ed., Oxford University Press, 1999.

Saturated Alkyl

A saturated alkyl, or saturated alkyl group, is a series of chemically bonded carbon atoms, with each carbon atom bonded to the maximum number of atoms (which for carbon, is four atoms). Thus each carbon atom in the series has four single bonded substituents. Double bonds do not exist in saturated alkyls. Examples of saturated alkyl groups include, but are not limited to, ethane, propane, cyclopropane, butane, and decane.

Unsaturated Alkyl

An unsaturated alkyl is a series of chemically bonded carbon atoms where one or more of the carbon atoms is not bonded to the maximum number of atoms possible for carbon. Consequently one or more of the carbon atoms is bonded to another atom via a double or triple bond. For example, ethylene, propylene, and butylene are unsaturated alkyls.

Aromatic

Generally, groups with a cyclic structure having alternating double and single bonds can be identified as aromatic, using the Hückel rule. This rule states that if the number of electrons corresponding to double bonds and heteroatoms having available π electrons is 4n+2, where n is an integer (such as 0, 1, 2, 3, . . . ), then such a compound is aromatic. For example, benzene with six delocalized electrons, has n=1 (4n+2=6 delocalized electrons) and is considered aromatic. Aromatic compounds include, but are not limited to benzene, naphthalene, anthracene, pyridine, pyrrole, furan and thiophene.

Aromatic groups are aromatic compounds having a cyclic structure that are single or double bonded to another moiety. They may have mono-cyclic structures, such as benzene; bicyclic structures, such as naphthalene; or multi-cyclic structures, such as anthracene. As defined herein and in the appended claims, a cyclic structure includes mono-cyclic, bicyclic, and multi-cyclic structures.

Aromatic groups may have heteroatoms incorporated into their cyclic structures, such as furan, or be substituted with heteroatoms or carbon-containing substituents, such as phenol or a methyl substituted benzene. Thus, a heterosubstituted aromatic is an aromatic compound, which has a heteroatom incorporated in its cyclic structure or an attached hetero-atom containing substituent. Substituted aromatics have substituents attached to their cyclic structures.

For example a heterosubstituted aromatic multi-cyclic structure is

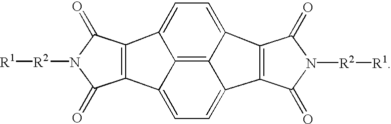

This representative aromatic compound has 6 cyclic structures with two of the cyclic structures being heterosubstituted. Additionally, the compound has R¹ and R² substituents, in addition to carbonyl substituents. As later defined, the carbonyl carbons, nitrogen atoms, all carbons that make up the cyclic structures, and R¹ and R² are in the backbone of the compound. Only the carbonyl oxygens are not in the backbone of the compound.

Heterosubstituted Unsaturated Alkyl

A heterosubstituted unsaturated alkyl is a series of chemically bonded carbon atoms, which do not have the maximum number of bonds, and are also intermittently substituted with hetero-atoms. Since these alkyl groups are unsaturated, there will be double or triple bonds between various carbon atoms. Hetero-atoms are defined as atoms other than carbon. Examples of hetero-atoms include, but are not limited to, nitrogen, oxygen, sulfur, and halides. Examples of heterosubstituted unsaturated alkyls include, but are not limited to, chloro-ethane, 1-amino-propane, and 1-butanol.

Heterosubstituted Saturated Alkyl

A heterosubstituted saturated alkyl is a series of chemically bonded carbon atoms, which have the maximum number of bonds, and are also intermittently substituted with hetero-atoms. Examples of heterosubstituted saturated alkyls include, but are not limited to, chloro-ethane, 1-amino-propane, and 1-butanol.

Halogen

Halogens are fluorine, chlorine, bromine, and iodine. Halides are halogens in a ⁻1 formal oxidation state. It should be understood that the terms halogen and halide are used interchangeably in the specification and appended claims to refer to the circumstances when a halogen is bonded to other atoms.

For example, a halogen or halide containing moiety is any molecule that includes a combination of other atoms to which a halogen or halide group is attached or incorporated. Examples of halide containing moieties include, but are not limited to, —C(O)Cl, —OCl, benzyl chloride, and

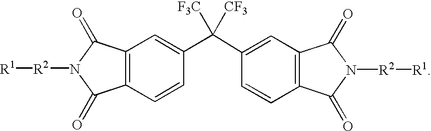

Thiophene Containing Moiety

A thiophene containing moiety is a molecular entity to which a thiophene moiety, C₄H₄S, is attached or incorporated. One or more hydrogen atoms may be removed from the thiophene moiety when attached or incorporated. While any thiophene containing moiety may be used that is compatible with NLO polymer synthesis, thiophene containing moieties with the structure

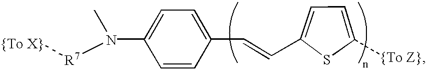

wherein n is an integer from 1 to 10, and R⁷ is a saturated or unsaturated alkyl, an aromatic, a substituted aromatic, a heterosubstituted unsaturated or saturated alkyl, or a heterosubstituted aromatic; or

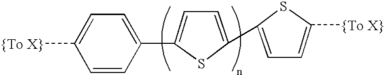

wherein n is an integer from 0 to 10, are preferred.

Carbonyl Containing Moiety

A carbonyl containing moiety is any combination of other atoms to which a carbonyl group (—C(O)—) is attached or incorporated. Examples of moieties incorporating carbonyl groups include, but are not limited to, —C(O)OH, —C(O)OCH₃, —C(O)Cl,

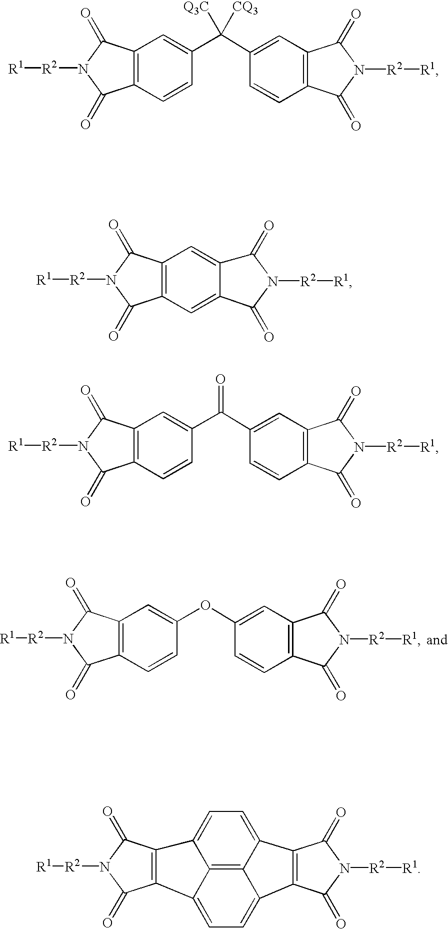

Labile Group

As used in the specification and appended claims, labile groups are defined as transitory molecular entities, or groups, which can be replaced with other molecular entities under specified conditions to yield a different functionality. Preferably, one or more labile groups are removed from the monomers when polymerized.

Examples of labile groups include, but are not limited to protons (—H), hydroxyl groups (—OH), alkoxy groups (—OR), and halogens (—X), such as fluorine, chlorine, bromine, and iodine. Labile groups may be attached to other molecular entities, including, but not limited to, aromatic and substituted aromatic cyclic structures, oxygen containing moieties, carbonyl containing moieties, and thiophene containing moieties, or mixtures thereof.

Nonlinear Optic Materials

Nonlinear optic materials are those that demonstrate non-linear optic effects when irradiated with light. Nonlinear optic polymers contain nonlinear optic chromophores that provide the polymer with its nonlinear optic character. The overall nonlinear optic character of the NLO polymer matrix is mostly determined by the type of NLO chromophore incorporated into the polymer, however, the polymer backbone to which the chromophores are attached, and the matrix structure of the polymer in the device can also affect the NLO performance of the material.

### Description

The present invention relates to polymeric, nonlinear optical materials, their methods of synthesis, and devices in which they are useful. The disclosed NLO polymers may be synthesized under mild conditions. The NLO polymers preferably contain nonlinear optic chromophores covalently bonded as side-chains to polymeric backbones. The polymeric backbones can contain esterimide or other functionality, preferably imparting high temperature stability to the NLO polymers. The backbones may also be crosslinked to increase the dipole stability of the resultant polymers.

In a preferred aspect, the disclosed synthetic methods provide a system to covalently bond NLO chromophores to a polymer backbone. Thus, a NLO polymer can result that demonstrates high thermodynamic stability and uniform composition. While not wishing to be bound by any particular theory, it is believed that high thermodynamic stability is provided by the backbone, while the uniformity of the covalently bonded functionalized polymers provides lowered scattering loss. In addition, absorption losses may be reduced through partially or substantially deuterating the monomer units and/or the linking monomers. Many different NLO chromophores may be bonded to a wide variety of polymer backbones using the disclosed methods. Thus, large optical nonlinearity may be provided through chromophore selection.

In relation to conventional NLO polymers, NLO polymers in accord with the present invention surprisingly achieve one or more of the following features: high temporal stability of dipole orientation, large optical nonlinearity, minimum optical loss, and the ability to be processed at high temperature (high T_{g}). A high T_{g} temperature is preferably defined as 150° C. and above and more preferably as 170° C. and above. In an especially preferred aspect, high T_{g} temperature is 200° C. and above. These characteristics make the NLO polymers of the present invention especially suited to high speed switching applications due to their low dielectric constants and the excellent match between their rf and optical indices.

The high T_{g} temperatures can also provide the benefits of easier fabrication and significant lifetimes for devices incorporating the NLO polymers. In a preferred aspect, high molecular weight (MW) NLO polymers are synthesized that provide enhanced mechanical strength and lower optical loss in relation to conventional NLO polymers.

In accordance with the present invention, multiple monomer units are used to synthesize the polymeric backbone of the NLO polymers. In one aspect, the monomers units are NLO monomers that include nonlinear optic chromophores. As used in the following specification and appended claims, these monomers have the formula X-Y-Z, where X forms the “head” of the monomer unit; Y is an electron donating group; and Z is an electron withdrawing group. In combination, the Y and Z groups constitute a side-chain, or “tail,” that forms the NLO chromophore portion of the NLO chromophoric monomer. While not required, a spacer moiety may be included between the X head group and the Y—Z tail group.

While the X groups of the NLO chromophoric monomers may be directly polymerized to form the backbone of a NLO polymer, preferably, linking monomers are polymerized with the X groups to form the backbone. As referred to in the specification and appended claims, linking monomers are monomer units that may be used to attach the head groups of NLO monomers to form the backbones of NLO polymers. Especially preferred crosslinkable linking monomers may be crosslinked to join the backbones of the NLO polymers.

Polymer Backbone

The backbone of a NLO polymer is formed from multiple monomers or monomer units that are covalently linked in a series. As defined in the specification and appended claims, groups or moieties that reside in the backbone of a polymer contain atoms that one or more lines that follow the covalent bonds and that start at one end of the polymer and end at the other end of the polymer may be drawn through, without reverse. Thus, in the following illustrative polymer structure, all the C atoms with superscripts are in the backbone of the polymer. Side-chains, such as the —OCH₂CH₂OH group, and substituents, such as the —OH group and the oxygen of the carbonyl are excluded from the backbone of the illustrative polymer.

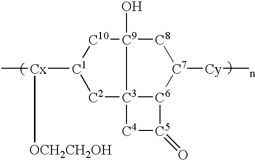

Atoms Cx, Cy, and C¹ through C⁷ are in the backbone because a line that follows the bonds starting at Cx and terminating at Cy passes through C¹ through C⁷, without reverse. Likewise, C⁸ through C¹⁰ are in the backbone of the polymer because a second line that follows the bonds starting at Cx and terminating at Cy passes through them, in addition to C¹ and C⁷, which were already determined to be in the backbone, without reverse. However, none of the O atoms are in the backbone, because a line following the bonds that starts at Cx and ends at Cy cannot pass through any of the O atoms without reverse.

In one aspect, polymer backbones include polyester imide functionality. Polyester imide functionality is defined as a series of imide and ester (—C(O)O—) groups, which are linked in series. A representative NLO monomer that includes polyester imide functionality is pictured below, where X includes an aromatic group with at least one single bond attachment to at least one of the adjacent oxygen atoms, Y and Z in combination form a NLO chromophore, and A is a group including imide functionality. In the representative high molecular weight NLO monomer pictured below, n can be an integer from 1 to 50,000, preferably an integer from 1 to 5,000, and more preferably an integer from 1 to 1,000. At present, an especially preferred value for n is an integer from 1 to 100.

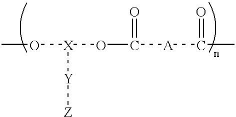

Preferable aromatic groups, for incorporation at —X— include, but are not limited to, substituted and unsubstituted benzene, substituted and unsubstituted heterocycles, substituted and unsubstituted cyclic structures, and substituted and unsubstituted hetero-cyclic structures. Especially preferred aromatic groups for incorporation at —X— include

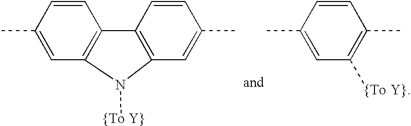

By imide functionality, it is meant a group that is a nitrogen analogue of an anhydride. While many methods are known to those of ordinary skill in the art to synthesize imides, they are often formed by the exchange of ammonia or amines with anhydrides, or by the reaction of amides with carboxylic acids. While A can be any group with imide functionality that is compatible with NLO polymer synthesis, groups with the structure

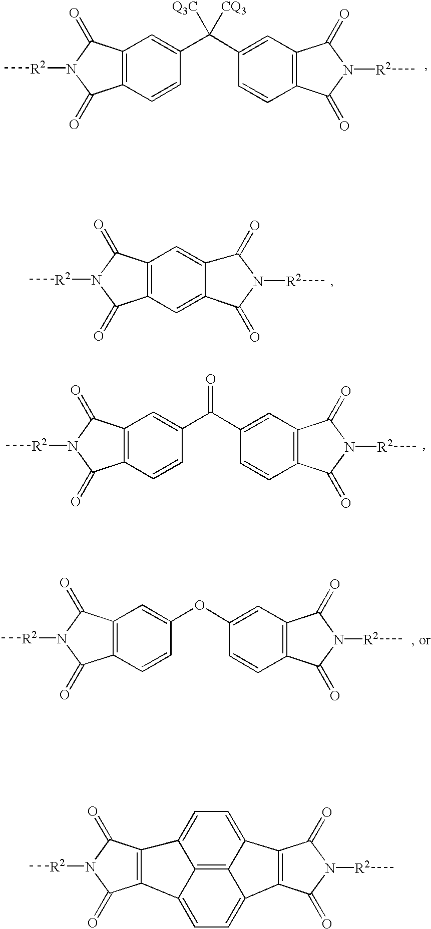

are preferred.

In these preferred groups having imide functionality, Q is preferably a halogen and R² is preferably a single bond, saturated alkyl group, unsaturated alkyl group, heterosubstituted saturated alkyl group, heterosubstituted unsaturated alkyl group, heterosubstituted aromatic group,

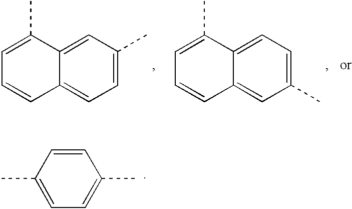

R²groups having the structure

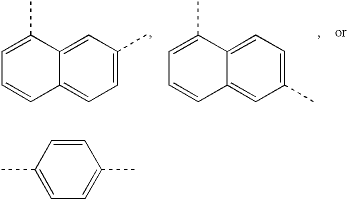

are especially preferred at present.

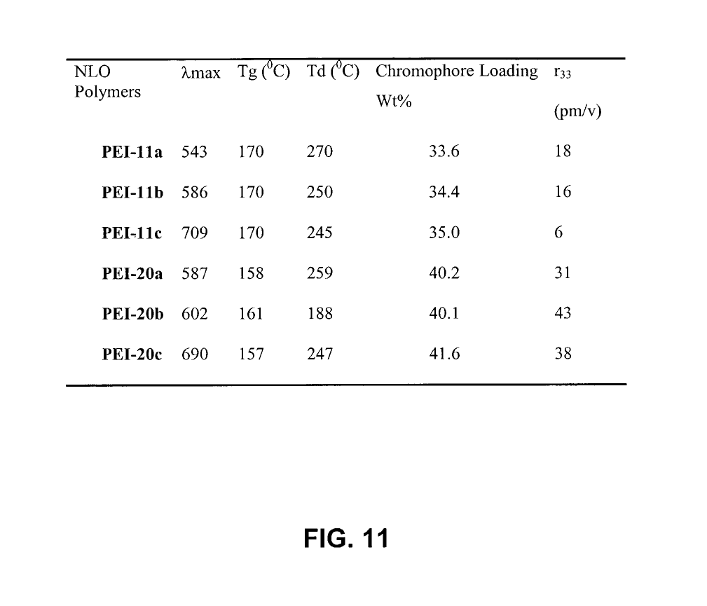

In a preferred aspect, polyester imides are directly synthesized from carboxylic acids containing imide moieties and phenols. In another preferred aspect, imide functionality is introduced into dicarboxylic acid monomers. As can be seen in FIG. 11, NLO polymers in accord with the present invention have high glass transition temperatures, while demonstrating preferable r₃₃ values.

High glass transition temperature (T_{g}) is defined as 145° C. are higher, more preferably about 150° C. and higher, and even more preferably about 160° C. and higher. In an especially preferred aspect, NLO polymers in accord with the present invention have high glass transition temperatures of about 170° C. and higher. Preferable r₃₃ values are about 10 and higher, more preferably about 15 and higher, and even more preferably about 30 and higher. In an especially preferred aspect, r₃₃ values are about 38 and higher.

Due to the exponential relationship between the glass transition temperature (T_{g}) of the NLO polymer used in an electro-optical device and the life expectancy of the resultant device, even small increases in T_{g} result in significant increases in the useful life of the device. This is demonstrated in the table below, which shows the estimated useful half-lives of EO devices as a function of operating temperature for a NLO polymer having a T_{g} of about 140° C.

| Operating Temperature in ° C. | Lifetime in Years |
|---|---|
| 20 | 1 × 10¹¹ |
| 40 | 9 × 10⁶ |
| 60 | 2 × 10⁴ |
| 80 | 19 |
| 100 | 0.05 |

A more complete discussion of the relationship between T_{g} and useful lifetimes of EO devices may be found in M. Stahelin, et al., Re-evaluation of the thermal stability of optically nonlinear polymeric guest-host systems, Appl. Phys. Lett. 61 (14)1626 (1992) and G. R. Mohlmann, Polymeric optically nonlinear x(2) waveguide devices, in Organic materials for photonics, 253-276, 262 (G. Zerbi, ed., 1993), incorporated by reference in their entirety, except that in the event of any inconsistent disclosure or definition from the present application, the disclosure or definition herein shall be deemed to prevail.

While aromatic polyesters allow for ease of synthesis and acceptable r₃₃ values, they generally have low glass transition temperatures between 80 and 120° C. Thus, their useful life in EO devices is severely limited. In contrast to polyesters, aromatic polyimides can have high glass transition temperatures from 200 to 240° C., but are difficult to synthesize and have low r₃₃ values, making their EO performance unacceptable. Surprisingly, the NLO polymers in accord with the present invention are easily prepared and have high T_{g} and preferable r₃₃ values.

Chromophoric Monomer Units

Preferably, the backbones of NLO polymers include chromophoric monomers having the structure X—Y—Z. In one aspect, multiple X monomers, with their attached —Y—Z side-chains, are directly polymerized to form the backbone of the NLO polymer. In combination, —X—Y form the nonlinear optic chromophore portion of the chromophoric monomers.

Preferable X moieties include carbazole

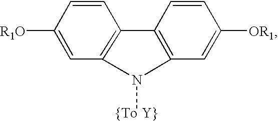

wherein the nitrogen atom is single bonded to an electron donating group Y or to a spacer moiety that is bonded to an electron donating group Y; phenol

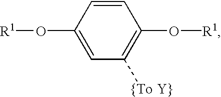

wherein the cyclic structure of the benzene ring is single bonded to the electron donating group Y or to a spacer moiety that is bonded to an electron donating group Y; and amine

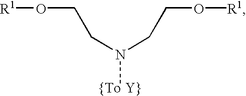

wherein the nitrogen atom is single bonded to the electron donating group Y or to a spacer moiety that is bonded to an electron donating group Y. In each chromophoric monomer, R¹ contains a labile group. In an especially preferred embodiment, R¹ is a hydrogen atom.

Nonlinear optic chromophores (—Y—Z from above) are defined as portions of a molecule that create a nonlinear optic effect when irradiated with light. The chromophores are any molecular unit whose interaction with light gives rise to the nonlinear optical effect. The desired effect may occur at resonant or nonresonant wavelengths. The activity of a specific chromophore in a nonlinear optic material is stated as their hyper-polarizability, which is directly related to the molecular dipole moment of the chromophore.

Many useful NLO chromophores are known to those of ordinary skill in the art. While any NLO chromophore that provides the desired NLO effect to the NLO polymer and is compatible with the synthetic methods used to form the NLO polymer may be used, preferred NLO chromophores include an electron donating group and an electron withdrawing group, as further defined below. More preferred are NLO chromophores that include an electron donating group and an electron withdrawing group connected by a conjugated series of bonds.

To determine if a material, such as a compound or polymer, includes a nonlinear optic chromophore, the following test may be performed. First, the material in the form of a thin film is placed in an electric field to align the dipoles. This may be performed by sandwiching a film of the material between electrodes, such as indium tin oxide (ITO) substrates, gold films, or silver films, for example.

To generate a poling electric field, an electric potential is then applied to the electrodes while the material is heated to near its glass transition (T_{g}) temperature. After a suitable period of time, the temperature is gradually lowered while maintaining the poling electric field. Alternatively, the material can be poled by corona poling method, where an electrically charged needle at a suitable distance from the material film provides the poling electric field. In either instance, the dipoles in the material are believed to align.

The nonlinear optical property of the poled material is then tested as follows. Polarized light, often from a laser, is passed through the poled material, then through a polarizing filter, and to a light intensity detector. If the intensity of light received at the detector changes as the electric potential applied to the electrodes is varied, the material incorporates a nonlinear optic chromophore and has an electro-optically variable refractive index. A more detailed discussion of techniques to measure the electro-optic constants of a poled film that incorporates nonlinear optic chromophores may be found in Chia-Chi Teng, Measuring Electro-Optic Constants of a Poled Film, in Nonlinear Optics of Organic Molecules and Polymers, Chp. 7, 447-49 (Hari Singh Nalwa & Seizo Miyata eds., 1997), incorporated by reference in its entirety, except that in the event of any inconsistent disclosure or definition from the present application, the disclosure or definition herein shall be deemed to prevail.

The relationship between the change in applied electric potential versus the change in the refractive index of the material may be represented as its EO coefficient r₃₃. This effect is commonly referred to as an electro-optic, or EO, effect. Devices that include materials that change their refractive index in response to changes in an applied electric potential are called electro-optical (EO) devices.

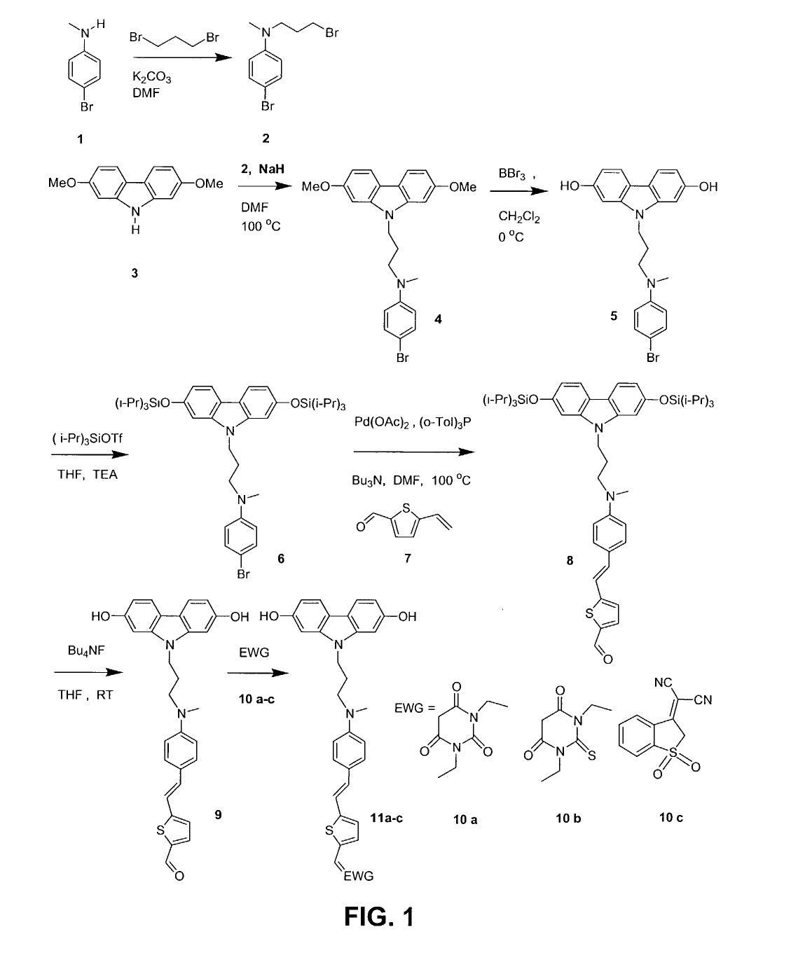

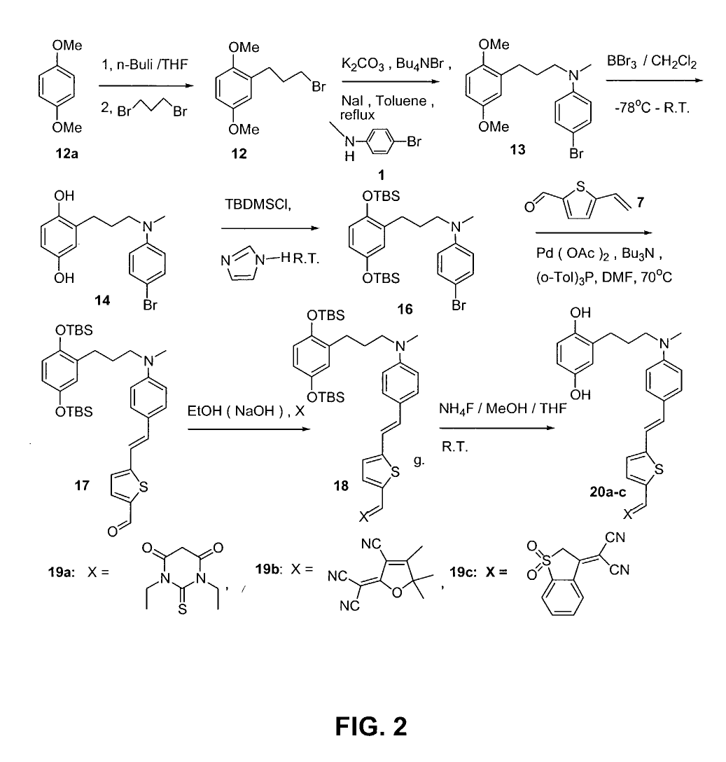

Especially preferred NLO chromophores in accordance with the present invention are those of the “push-pull” type, for example as shown bonded to head group 3 in FIG. 1 as 11a-c, and bonded to head group 12 in FIG. 2 as 20a-c. Although different electron donating groups (EDGs) may be used, these exemplary chromophores each have an amino containing group that donates electrons and different electron withdrawing groups (EWGs), for example 10a-c in FIG. 1 and 19a-c in FIG. 2. Different EWGs allow for the μβ values and thermal stability of the resultant NLO chromophores to be varied.

The μβ values of chromophores in NLO chromophoric monomers 11a, 11b, and 11c in FIG. 1 are about 1200×10⁻⁴⁸ esu, about 2400×10⁻⁴⁸ esu, and about 5000×10⁻⁴⁸ esu, respectively. A more detailed discussion of μβ values and their use in evaluating the EO performance of various NLO chromophores can be found in Shu, C. F. et al. Chem. Commn. 1996, 2279 and Sun, S. S. et al. Chem. Mater. 1996, 8, 2539, incorporated by reference in its entirety, except that in the event of any inconsistent disclosure or definition from the present application, the disclosure or definition herein shall be deemed to prevail.

Electron donating groups (—Y— from above) are defined as molecular entities, or groups, that can transfer electron density to another molecular entity or group. While any electron donating group may be used that is compatible with NLO polymer synthesis and provides a desirable EO in combination with the chosen electron withdrawing group, electron donating groups with the structure

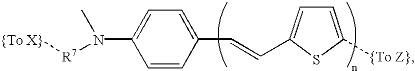

wherein n is an integer from 1 to 10, and R⁷ is a saturated or unsaturated alkyl, aromatic, heterosubstituted unsaturated or saturated alkyl, or heterosubstituted aromatic; or

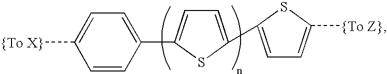

wherein n is an integer from 0 to 10, are preferred. Electron donating groups having the structure

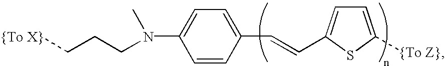

wherein n is 2 are especially preferred at present.

An electron withdrawing group (—Z from above) is any group that can withdraw electron density from another group, or molecular entity. While any electron withdrawing group may be used that is compatible with NLO polymer synthesis and provides a desirable EO in combination with the chosen electron donating group, electron withdrawing groups with the structure

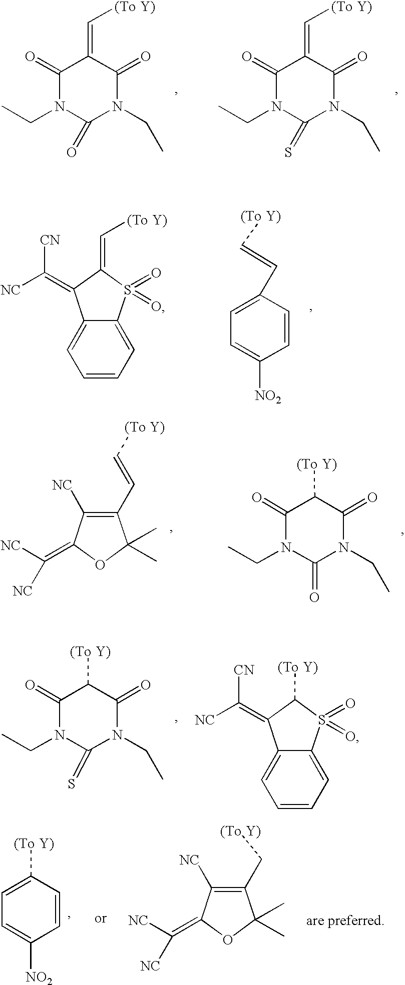

Linking Monomers

In a preferred aspect, one or more linking monomers link the X—Y—Z chromophoric monomers to form the backbone of the NLO polymer. Preferably, when a chromophoric monomer and a linking monomer polymerize or link, at least one labile group is lost from the X monomer and at least one labile group is lost from the linking monomer. The loss of the two labile groups creates open bonding sites, thus allowing the monomers to link.

In a preferred aspect, linking monomers, in combination with the X portion of the chromophoric monomers, form the NLO polymer backbone. The linking monomers can include cyclic aromatic groups, esters, and imides, for example. Examples of preferable linking monomers include, but are not limited to,

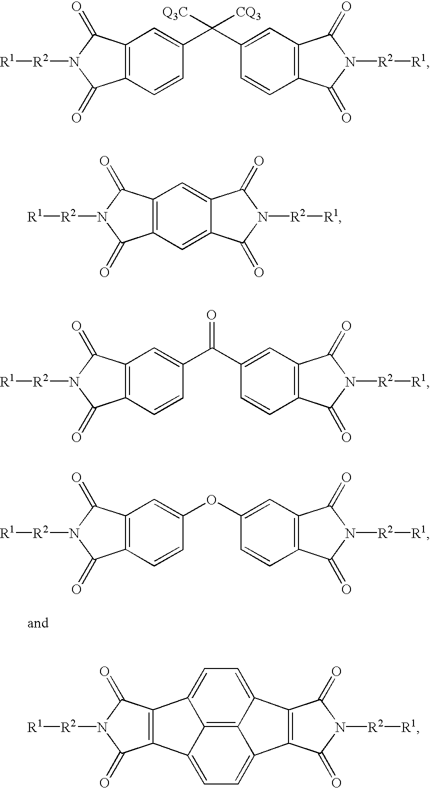

wherein R¹ contains a labile group and Q is a halogen.

Preferably for these linking monomers, R² can be a single bond, a carbonyl containing moiety, a saturated or unsaturated alkyl group, an aromatic group, a heterosubstituted saturated or unsaturated alkyl group, a heterosubstituted aromatic group, and any combination thereof. In an especially preferred aspect, R² is

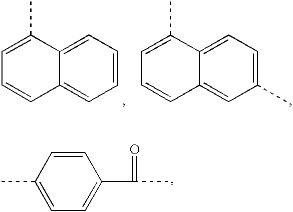

and mixtures thereof.

Another preferred type of linking monomer is referred to as a crosslinkable linking monomer because it incorporates a crosslinking substituent, which can undergo crosslinking. In one aspect, crosslinkable linking monomers may be polymerized with any compatible monomer unit that includes a nonlinear optic chromophore. When crosslinked, crosslinkable linking monomers preferably provide a high thermal stability in dipole orientation to the resultant NLO polymer. This high thermal stability may be provided through crosslinking of the crosslinkable linking monomers on different polymer backbones.

An especially preferred crosslinkable linking monomer includes the following structure

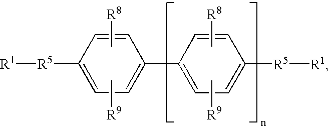

wherein R¹ contains a labile group; R⁵ preferably includes a single bond, an oxygen atom, a carbonyl group, a carbonyl containing moiety, or a thiophene containing moiety; R⁸ includes a crosslinking substituent; and R⁹ is a hydrogen atom, a crosslinking substituent, or a nonlinear optic chromophore, such as —Y—Z from above. The bonds from R⁸ and R⁹ may terminate at either carbon they are drawn between. Preferably, n is an integer from 1 to 100, and more preferably n is an integer from 1 to 50. In an especially preferred crosslinkable linking monomer, n is from 1 to 5.

Another especially preferred crosslinkable linking monomer includes the following structure

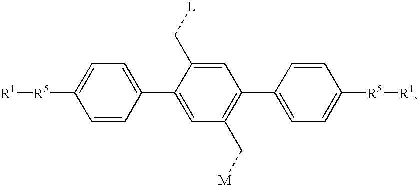

wherein R contains a labile group; R⁵ preferably includes a single bond, an oxygen atom, a carbonyl group, a carbonyl containing moiety, or a thiophene containing moiety; L is a crosslinking substituent; and M is the same as L or is a nonlinear optic chromophore, such as —Y—Z from above. Either crosslinkable linking monomer is especially preferred at present when R⁵ is an oxygen atom, ester, or carboxylic acid group.

A substituent capable of undergoing radical crosslinking, or a crosslinking substituent, is a substituent that can serve to chemically bond two or more strands of NLO polymers together via a crosslinking reaction. Thus, when a first crosslinking substituent on a first linking monomer and a second crosslinking substituent on a second linking monomer crosslink, the first and second linking monomers are crosslinked. While cyclization type crosslinking, such as by a [2+2] reaction, light initiated radical crosslinking, and other methods known to those of ordinary skill in the art can be used, thermally initiated radical crosslinking is especially preferred at present.

Preferable crosslinking substituents include, but are not limited to, moieties containing the structure

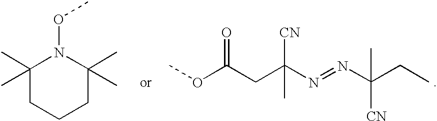

Thus, either of these substituents, for example, may be the L group on the above linking monomer. Either of these, and other crosslinking substituents, can generate highly reactive radicals that serve to crosslink the polymers when thermally excited. Radicals are defined as atoms or groups that possess an unpaired electron.

By crosslinking the backbones of the NLO polymers using crosslinking substituents, the dipole orientation of the resultant crosslinked NLO polymers can be fixed. Thus, the temporal stability of the NLO effect can be enhanced. While not wishing to be bound by any particular theory, such crosslinking is believed to reduce the motion of the individual NLO polymers, which make up the polymer matrix.

Especially preferred NLO polymers have a crosslinking temperature that is higher than the glass transition temperature (T_{g}) of the NLO polymer. Preferably, thermal initiation of the radical crosslinking reaction is performed at a temperature that is high enough to align the dipole of the NLO chromophore, but lower than the temperature at which the NLO chromophore begins to decompose.

The crosslinkable NLO polymers may be crosslinked before, during, or after poling with the electric field to align the dipoles. In an especially preferred embodiment, the NLO polymers are crosslinked before poling. While not wishing to be bound by any particular theory, it is believed that the temperature necessary for poling (near the T_{g}) does not adversely affect the previously crosslinked polymers. Preferably, a more stable poled polymer film, with glass transition temperatures of about 170° C. and higher, can be obtained by crosslinking the linking monomers prior to poling.

Preferable chromophoric and/or linking monomers that are used to synthesize NLO polymers may be deuterated. To form a partially deuterated monomer, one or more of the hydrogen atoms covalently attached to the monomer are replaced with deuterium atoms. To form a substantially deuterated monomer, at least half of the hydrogen atoms covalently attached to the monomer are replaced with deuterium atoms. In a preferred aspect, the chromophoric and/or linking monomers, which form the NLO polymer, are partially deuterated. In an especially preferred aspect, the chromophoric and/or linking monomers, which form the NLO polymer, are substantially deuterated.

By replacing the lighter hydrogen atoms with heavier deuterium atoms, the light energy lost due to absorption by the C—H bonds in the infrared region (vibrational transitions) may be reduced. By partially or substantially deuterating the NLO polymers (replacing C—H functional groups with C-D functional groups), the vibrational absorption frequencies of the resultant polymer matrix can be shifted. A more detailed discussion of using heavier atoms to replace hydrogen atoms in NLO materials may be found in Brauer, et al., Polymers for passive and switching waveguide components for optical communication, in Polymers in Optics: Physics, Chemistry, and Applications, 338-40 (Roger A. Lessard & Werner F. Frank eds., 1996), incorporated by reference in its entirety, except that in the event of any inconsistent disclosure or definition from the present application, the disclosure or definition herein shall be deemed to prevail.

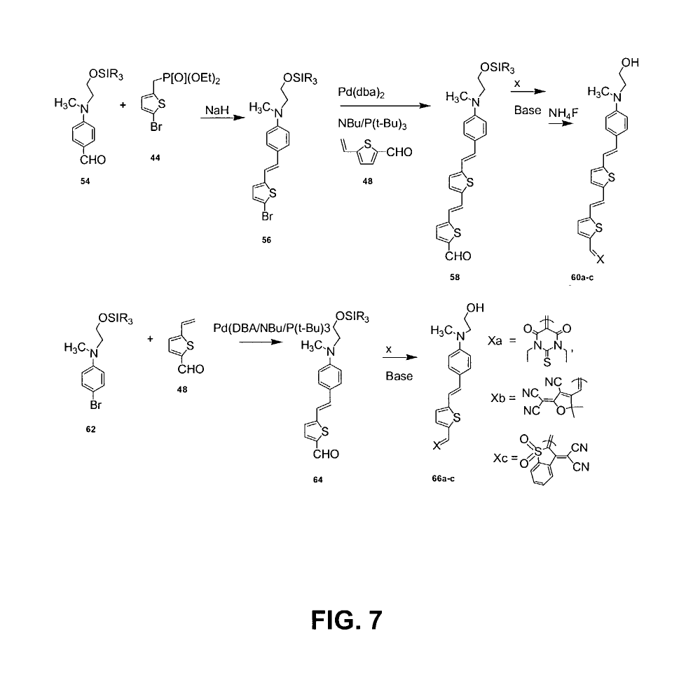

For example in FIG. 7, if compound 54 is synthesized from deuterated benzene (benzene-d₆; a common NMR solvent available from Aldrich, Milwaukee, Wis.) and/or one or more of the hydrogens on thiophene compounds 44 and 48 are also replaced with deuterium, such as by using thiophene-d₄ as a starting material, a deuterated chromophore 60a-c results.

Electro-Optical Devices

When incorporated into an electro-optical device, NLO polymers, including chromophoric monomers and optional linking monomers, form a matrix. Crosslinked or non-crosslinked NLO polymers can form many types of polymer matrices when incorporated into an EO device. For example, the polymer may be suspended in a solution or dispersion and cast as a film on a substrate. Preferable film casting processes include, but are not limited to, spin coating, spraying, and Langmuir-Blodgett deposition. Upon drying, a polymer matrix can form. Such films can be patterned with many techniques, including, but not limited to, ion/plasma etching and photolighographic processing. Many processes are known to those of ordinary skill in the art to form polymer matrices from NLO polymers in accord with the present invention.

In a preferred embodiment, NLO polymers are applied to substrate materials utilized in optical devices. The substrate material may be an inorganic, which includes, but is not limited to silicon, silicon dioxide, gallium arsenide, or gallium aluminum arsenide. Silicon, silicon wafers, or silicon coated onto glass, plastic, or metal are especially preferred substrates.

NLO polymers may also be formed into a matrix as a bulk substance that can be machined into a desired shape or drawn or extruded into fibers. The polymers may also be made into devices by injection molding, press printing, and special inkjet printing, for example.

In accord with the present invention, NLO polymers may be used in many electro-optical devices (the terms device, optical device, and electro-optical device are used interchangeably), including, but not limited to, passive and active waveguides, directional couplers, optical flip-flop devices, devices made from bulk material, and photoconductive films. Preferable waveguide type devices made from NLO polymers can be either passive devices; which include, but are not limited to, beam splitters; or active devices; which include, but are not limited to, phase modulators and Mach-Zehnder modulators. Preferable modulator type devices include straight channel, phase, and intensity modulators.

Preferable active devices also include optical switches and electro-optically controlled tunable optic filters. In one aspect, these filters operate by changing the refractive index by the EO effect. Preferable passive waveguide devices include, but are not limited to, arrayed waveguide gratings (AWG), optical add/drop modules (OADM), and optical interconnects for on-chip integration.

Preferable optical devices in accord with the present invention in which NLO polymers are especially useful include electro-optical modulators having a Mach-Zehnder interferometer design, which preferably consists of an upper cladding polymer layer, a NLO polymer layer, and a lower cladding polymer layer. Another especially useful application for NLO polymers are phase modulators having a single channel design, which preferably consists of an upper cladding polymer layer, an NLO polymer layer, and a lower cladding polymer layer.

A more complete discussion of electro-optical modulators may be found in C. C. Teng, Traveling-wave polymeric optical intensity modulator with more than 40 GHz of 3-dB electrical bandwidth, Appl. Phys. Lett., vol. 60(13), 1538, 1992, incorporated by reference in its entirety, except that in the event of any inconsistent disclosure or definition from the present application, the disclosure or definition herein shall be deemed to prevail.

Alternate designs of electro-optical modulator devices, such as those with NLO polymer coated waveguides, are also especially preferred applications for the NLO polymers of the present invention. A more complete discussion of coated waveguide devices may be found in Y. Enami, et al., Poling of soda-lime glass for hybrid glass/polymer electro-optic modulators, Appl. Phys. Lett., vol. 76 (9), 1086, 2000, incorporated by reference in its entirety, except that in the event of any inconsistent disclosure or definition from the present application, the disclosure or definition herein shall be deemed to prevail.

Other preferable devices in accord with the present invention in which NLO polymers are especially useful include directional couplers, which preferably include an upper cladding polymer layer, an NLO polymer layer, and a lower cladding polymer layer. A more complete discussion of directional couplers devices may be found in D. An et al., Polymeric electro-optic modulator based on 1×2 Y-fed directional coupler, Appl. Phys. Lett., vol. 76 (15), 1972, 2000, incorporated by reference in its entirety, except that in the event of any inconsistent disclosure or definition from the present application, the disclosure or definition herein shall be deemed to prevail.

Another preferable device in accord with the present invention in which NLO polymers are especially useful is optical switches. In these devices, the NLO polymers can form a cascade of electro-optical modulators or directional couplers that work in concert to provide an optical switch for many applications, including telecommunication networks. In many aspects, these optical switches perform similar functions for light that transistors perform for electricity.

Synthetic Approach

Referring to the illustrative synthetic approach of FIG. 1, a preferred reaction sequence is shown for NLO monomers 11a-c. While other reaction sequences may be used, in this sequence, the sensitive NLO chromophore is added during the last reaction to reduce decomposition.

In FIG. 1, alkylation of 4-bromo-N-methylaniline 1 with 1,3-dibromopropane yields compound 2. The 2,7-dimethoxycarbazole 3 (head) was alkylated with 2 in DMF in the presence of NaH to yield compound 4. Demethylation of 4 using BBr₃ gave the corresponding diphenol 5, which was protected by the triisopropyl sillyl group using (i-Pr)₃SiOTf to give compound 6. Aldehyde 8 was then generated by coupling compound 6 with 5-vinyl-2-thiophenecarbaldehyde 7 under the Heck reaction conditions. A more detailed discussion of the Heck reaction and its use in chemical synthesis is given in Heck, R. F., Org. React., 1982, 27, 345, incorporated by reference in its entirety, except that in the event of any inconsistent disclosure or definition from the present application, the disclosure or definition herein shall be deemed to prevail.

Removal of sillyl group from 8 with 1 M Bu₄NF in THF gave the corresponding diphenol monomer 9. A further Knoevenagle reaction of compound 9 with electron-withdrawing precursors 10a-c generated the corresponding monomers NLO chromophoric monomers 11a-c.

Referring to FIG. 2 a preferred reaction sequence for NLO chromophoric monomers 20a-c may include replacing the carbazole unit 3 from FIG. 1 with phenyl group 12a. The basic strategy for the syntheses of these monomers is preferably similar to that used in FIG. 1 for NLO chromophoric monomers 11a-c, except for the starting material.

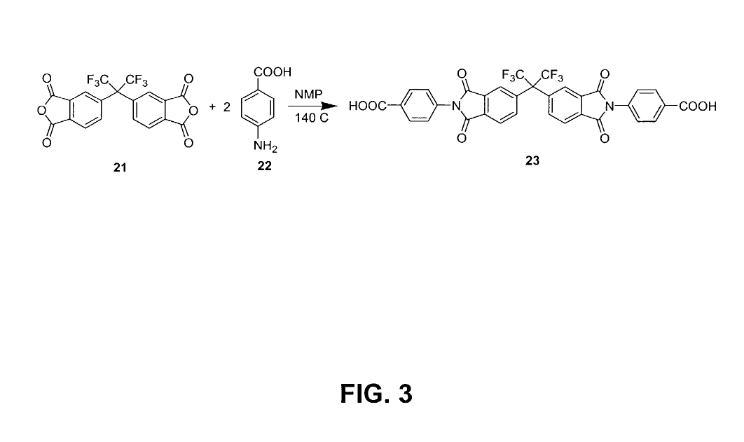

Referring to FIG. 3, a preferred reaction sequence for preparing linking monomer 23 may include reacting aminbbenzoic acid 22 with dianhydride 21 in a high boiling solvent, such as NMP at 140° C., for 12 hours. The corresponding amic acid thought to be formed in the early stages of the reaction (not shown), was believed to slowly cyclize via thermal imidization to the corresponding linking monomer 23.

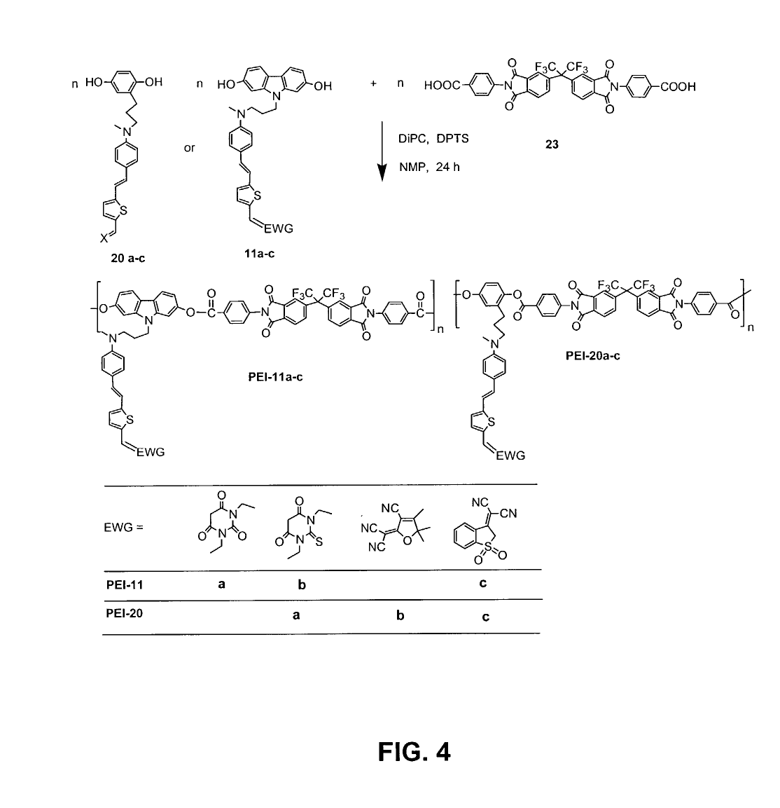

Referring to FIG. 4, a preferred reaction sequence for preparing NLO polymers PEI-11a-c and PEI-20a-c from NLO chromophoric monomers 11a-c and 20a-c can include polymerization with linking monomer 23, which includes imide functionality. Polyester imide (PEI) NLO polymers PEI-11a-c can result from NLO chromophoric monomers 11a-c, while polyester imide NLO polymers PEI-20a-c can result from NLO chromophoric monomers 20a-c.

NLO polyester imides are preferably synthesized from linking monomer 23, which was previously functionalized with carboxylic acids, and phenol functionalized NLO chromophoric monomers 11a-c and 20a-c. In one aspect, carbodiimide esterification conditions (1:1 molecular complex formed from 4-(dimethlamino) pyridine and p-toluenesulfonic acid (4-(dimethlamino)pyridinium 4-toluenesulfonate) DPTS), as shown in FIG. 4, was used. A more detailed discussion of carbodiimide esterification conditions and their use in polymerization may be found in Moore, J., et al. Macromolecules, 1990, 23, 65, incorporated by reference in its entirety, except that in the event of any inconsistent disclosure or definition from the present application, the disclosure or definition herein shall be deemed to prevail. Direct esterification is possible for NLO chromophoric monomer 20b and the corresponding carbonyl chloride of monomer 23.

Preferably, the polymerization reaction can be performed in anhydrous N-methyl-2-pyrolidone (NMP). In another aspect, other solvents known to one of ordinary skill in the art, including, but not limited to, N,N-dimethylformamide (DMF) and methylene chloride (CH₂Cl₂) may be used. The presence of the bulky hexafluoroisopropylidene moiety is believed to reduce intermolecular interactions and thus enhance polyester solubility in common organic solvents, including, but not limited to, THF and CHCl₃. In one aspect, the molecular weights of the resultant polymers, as determined by GPC using THF as eluent, can be in the range of 15-20 KDa, against polystyrene standards. One may preferably cast optical quality films of the polymers from tetrachloroethane (TCE) for NLO studies.

In another aspect, polymerization maybe carried under acidic conditions in heterogeneous media. In this aspect, an acidic surfactant, dodecylbenzenesulfonic acid (DBSA), can be used as both catalyst and water scavenger. Preferably, a solvent that is immiscible with water is used, such as, for example, toluene or halogenated hydrocarbons.

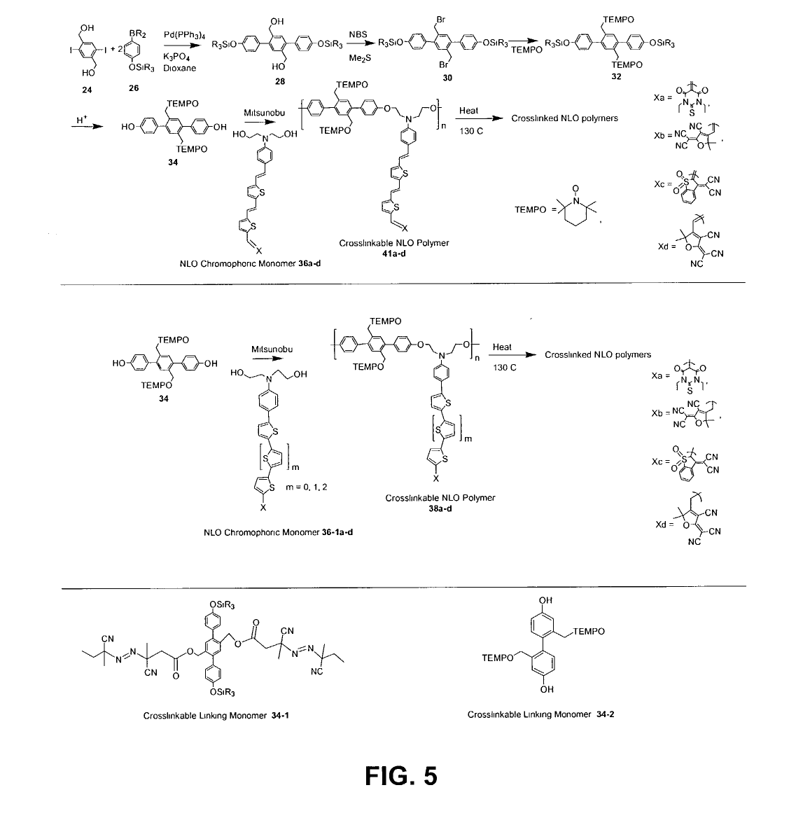

Referring to FIG. 5, a preferred synthetic approach to a thermally crosslinkable linking monomer embodying features of the present invention is shown. The halogen labile groups of diol 24 are reacted with 26 to give benzyl structure 28. After a crosslinking substituent, such as TEMPO, is added, a crosslinkable NLO polymer, such as 41a-d may be formed. Crosslinkable monomers 34, 34-1, and 34-2 are other examples of a preferred linking monomer with substituents capable of undergoing radical crosslinking. Crosslinkable linking monomers 34, 34-1, or 34-2 may then be reacted with NLO chromophoirc monomers, such as 36a-d or 36-la-d, to yield a crosslinkable NLO polymer 41a-d. Heat initiation may then be used to crosslink the polymers.

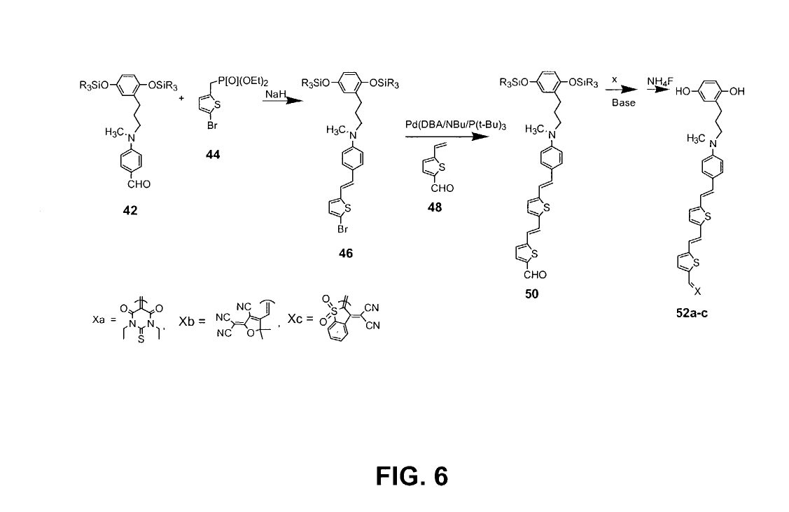

Referring to FIG. 6, a preferable synthetic method for forming the electron donating portion of a NLO chromophoirc monomer is shown. Compound 42 is first reacted with compound 44 and then 48 to yield the X—Y— portion 50 of a chromophoirc monomer. Electron withdrawing groups Xa-c (the Z portion of the chromophoirc monomer) may then be added to give complete X—Y—Z NLO chromophoirc monomers 52a-c.

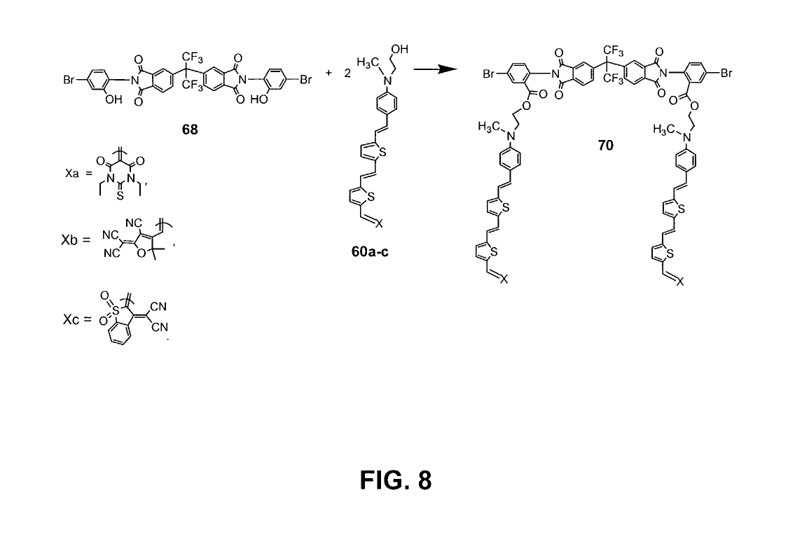

Referring to FIG. 7, NLO chromophoric monomers with tricyanofurane electron withdrawing groups are made. Compounds 54, 44, and 48 are reacted to give aldehyde 58 to which electron withdrawing groups Xa-c are attached to give NLO chromophores 60a-c. These chromophores may then be coupled to a polyamide backbone, such as 70, as shown in FIG. 8. A similar synthetic sequence can also produce NLO chromophores 66a-c, which may also be coupled to a polyamide backbone.

Referring to FIG. 8, a preferable synthetic method for forming a dihalogen type NLO chromophoric monomer 70 is shown. Two chromophores 60a-c are combined with a phenol derivatized linking monomer 68 to generate chromophoric monomer 70. Compound 70 may be directly polymerized, or polymerized with other moieties, such as linking monomers or crosslinkable linking monomers.

The preceding description is not intended to limit the scope of the invention to the preferred embodiments described, but rather to enable a person of ordinary skill in the art of polymer chemistry to make and use the invention. Similarly, the examples below are not to be construed as limiting the scope of the appended claims or their equivalents, and are provided solely for illustration. It is to be understood that numerous variations can be made to the examples below, which lie within the scope of the appended claims and their equivalents.

### EXAMPLES

Tetrahydrofuran (THF) was purified by distillation over sodium chips and benzophenone. NMP was purified by distillation over phosphorous pentaoxide. 4,4′(Hexafluoroisopropylidene)diphthalic anhydride was purified by recrystallization from acetic anhydride and dried in a vacuum at 150° C. All other chemicals were purchased from Aldrich Chemical Co., Milwaukee, Wis. and were used as received, unless otherwise stated.

Referring to FIG. 1, compound 1 (4-bromo-N-methylaniline), compound 3 (2,7-dimethoxy carbazole), compound 7 (5-vinyl-2-thiophenecarbaldehyde), (4-(dimethylamino)pyridinium-4-toluene-sulfonated), and compound 10c (3-(dicyanomethtylene)-2,3-dihydrobenzo[b]thiophene) were synthesized according to literature procedures known to those of ordinary skill in the art. Methods of synthesizing these compounds may be found in H. Saadeh, A. Gharavi, L. P. Yu, Macromolecules 1997, 30, 5403; S. Y. Yang, Z. H. Peng, L. P. Yu, Macromolecules 1994, 27, 5858; D. Yu, L. P. Yu, Macromolecules 1994, 27, 6718; D. Yu, A. Gharavi, L. P. Yu, Macromolecules 1995, 28, 784; D. Yu, A. Gharavi, L. P. Yu, J. Am. Chem. Soc. 1995, 117, 11680; D. Yu, A. Gharavi, L. P. Yu, Macromolecules 1996, 29, 6139; and Hsieh, B.; Litt, M. Macromolecules 1985, 18,1388; which are hereby incorporated by reference in their entirety, except that in the event of any inconsistent disclosure or definition from the present application, the disclosure or definition herein shall be deemed to prevail.

### Example 1

### Synthesis of Compound 2

A mixture of compound 1 (13.95 g, 75 mmol), 1,3-dibromopropane (22.70 g, 112.5 mmol), and K₂CO₃ (6.3 g, 112.5 mmol) in anhydrous DMF (75 mL) was heated at 95° C. for 2 hours. After cooling, the reaction mixture was treated with H₂O (200 mL) and extracted with CHCl₃ (3×80 mL). The combined extracts where washed with H₂O (4×100 mL) then dried over MgSO₄. After removal of the solvent the crude product was chromatographed on silica gel using CHCl₃:Hexane (8:2) to give compound 2 as a colorless oil (11.50 g, 50%).

¹H NMR (CDCl₃, ppm): δ 2.15 (t, J=6.5 Hz, 2H), 2.96 (s, 3H), 3.48 (m, 4H), 6.61 (d, J=9.1 Hz, 2H), 7.56 (d, J=9.1 Hz, 2H). Anal. Calcd for C₁₀H₁₃NBr₂: C, 39.12; H, 4.27; N, 4.56. Found: C, 38.93; H, 4.38; N, 4.59.

### Example 2

### Synthesis of Compound 4

To compound 3 (7.5 g, 33.04 mmol), dissolved in anhydrous DMF (150 mL) at 100° C. under nitrogen, was added NaH (1.2 g, 50.0 mmol). The resulted mixture was stirred at 100° C. for 2 hours and treated with compound 2 (11.2 g, 36.3 mmol) and then stirred at that temperature for another 2 hours. The reaction mixture was cooled and treated with H₂O (250 mL), and the precipitate was collected. The crude product was recrystallized from THF/MeOH to give compound 4 (13 g, 87%); mp 121-123° C.

¹H NMR (CDCl₃, ppm): δ 2.16 (t, J=7.2 Hz, 2H), 2.90 (s, 3H), 3.37 (t, J=7.5 Hz, 2H), 3.88 (s, 6H), 4.25 (t, J=7.1 Hz, 2H), 6.50 (d, J=9.1 Hz, 2H), 6.77 (d, J=2.1 Hz, 2H), 6.85 (dd, J=8.5, 2.2 Hz, 2H), 7.24 (d, J=9.1 Hz, 2H), 7.88 (d, J=8.5 Hz, 2H). Anal. Calcd for C₂₄H₂₅N₂O₂Br: C, 63.58; H, 5.56; N, 6.18. Found: C, 63.28; H, 5.56; N, 6.23.

### Example 3

### Synthesis of Compound 5

To a mixture of compound 4 (13.0 g, 28.7 mmol) in 100 mL distilled dichloromethane, boron tribromide (11.9 mL, 31.6 g, 126.3 mmol) was added dropwise at −78° C. After the mixture was stirred for 1 hour at −78° C. and 12 hours at about 25° C., the reaction mixture was chilled in an ice bath and hydrolyzed by adding MeOH dropwise. The white solid was collected, washed with water and then recrystallized from DMF/H₂O (12.0 g, 98%, mp 152-154° C.) to give compound 5.

¹H NMR (DMSO-d₆): δ 1.86 (m, 2H), 2.81 (s, 3H), 3.33 (t, J=7.4 Hz, 2H), 4.13 (t, J=7.1 Hz, 2H), 6.57 (m, 4H), 6.76 (dd, J=2.0 Hz, 2H), 7.2 (d, J=8.6 Hz, 2H), 7.66 (d, J=8.3 Hz, 2H). Anal. Calcd for C₂₂H₂₁N₂O₂Br: C, 62.13; H, 4.98; N, 6.59. Found: C, 62.53; H, 5.10; N, 6.50.

### Example 4

### Synthesis of Compound 6

A mixture of compound 5 (12.0 g, 28.23 mmol) and triethylamine (8.55 g, 11.80 mL, 84.7 mmol ) in THF (110 mL) was cooled to 0° C. and treated with (iPr)₃SiOTf (19.0 g, 16.6 mL, 62.1 mmol) dropwise. After the addition completed, the reaction mixture was allowed to warm to room temperature and then stirred at that temperature for 2 hours. The reaction mixture was filtered and the solvent was removed. The oil residue was chromatographed on silica gel using CHCl₃: Hexane (1:1) to give compound 6 a colorless oil (17.2 g, 83%); mp 48-50° C.

¹H NMR (CDCl₃, ppm): δ 1.10-1.25 (m, 42H), 2.07 (m, 2H), 2.80 (s, 3H), 3.29 (t, J=7.5 Hz, 2H), 4.16 (t, J=7.0 Hz, 2H), 6.42 (d, J=9.1 Hz, 2H), 6.73 (d, J=2.0 Hz, 2H), 6.75 (s, 2H), 7.18 (d, J=9.1 Hz, 2H), 7.76 (d, J=8.2 Hz, 2H). Anal. Calcd for C₄₀H₆₁N₂O₂Si₂Br: C, 65.10; H, 8.33; N, 3.80. Found: C, 64.84; H, 8.33; N, 3.66.

### Example 5

### Synthesis of Compound 8

A mixture of compound 6 (17.0 g, 23 mmol), compound 7 (3.84 g, 28 mmol), tri-o-tolylphosphine (1.41 g, 4.62 mmol), Bu₃N (21 mL) and Pd(OAc)₂ (0.21 g, 0.923 mmol) in DMF (80 mL) was heated at 90° C. for 12 hours. The reaction mixture was cooled and treated with H₂O (250 mL) and then extracted with CHCl₃ (3×100 mL). The organic extract was washed with H₂O (4×1 50 mL) and then dried over anhydrous MgSO₄. After the removal of the solvent, the residue was recrystallized from CHCl₃/Hexane to give compound 8 (12.9 g, 71%), mp 114-116° C.

¹H NMR (CDCl₃, ppm) δ 1.11 (m, 36H), 1.53 (m, 6H), 2.17 (m, 2H), 2.90 (s, 3H), 3.37 (t, J=7.0 Hz, 2H), 4.18 (t, J=6.8 Hz, 2H), 6.52 (d, J=8.7 Hz, 2H), 6.75 (m, 4H), 6.95 (d, J=16.0 Hz, ═CH, 1H), 6.97 (d, J=3.9 Hz, thienyl proton, 1H), 7.01 (d, J=16.0 Hz, ═CH, 1H), 7.28 (d, J=8.7 Hz, ArH, 2H), 7.61 (d, J=3.3 Hz, thienyl proton, 1H), 7.76 (d, J=8.1 Hz, 2H), 9.80 (s, aldehyde proton, 1H). Anal. calcd for C₄₇H₆₆N₂O₃SSi₂: C, 70.98; H, 8.36; N, 3.52. Found: C, 70.75; H, 8.16; N, 3.51.

### Example 6

### Synthesis of Compound 9

A solution of compound 8 (1.30 g, 1.64 mmol) in THF (50 mL) was treated with a solution of 1 M tetrabutylammonium florid in THF (2 mL). The resulted mixture was stirred under N₂ for 20 min. The reaction mixture was then treated with H₂O (50 mL) and then extracted with EtOAc (100 mL). The organic layer was dried over MgSO₄. After the solvent was removed the residue was recrystallized three times from THF/Hexane to give compound 9 as a red solid (0.65 g, 83%); mp 120-122° C.

¹H NMR (CDCl₃, ppm): δ 2.13 (m, 2H), 2.88 (s, 3H), 3.37 (t, J=7.1 Hz, 2H), 4.16 (t, J=6.8 Hz, 2H), 6.65 (d, J=8.7 Hz, 2H), 6.72 (m, 4H), 6.94 (d, J=16.0 Hz, ═CH, 1H), 7.01 (d, J=16.0 Hz, ═CH, 1H), 7.04 (d, J=3.9 Hz, thienyl proton, 1H), 7.24 (d, J=8.0 Hz, ArH, 2H), 7.61 (d, J=3.3 Hz, thienyl proton, 1H), 7.72 (d, J=8.7 Hz, 2H), 8.15 (s, OH, 2H), 9.79 (s, aldehyde proton, 1H). Anal. calcd for C₂₉H₂₆N₂O₃S: C, 72.17; H, 5.42; N, 5.80. Found: C, 70.82; H, 5.70; N, 5.29

### Example 7

### Synthesis of Monomers 11a, 11b, and 11c from FIG. 1

To a mixture of compound 9 (0.40 g, 0.71 mmol) in EtOH (5.0 mL), was added compound 10a (0.16 g, 0.85 mmol). The reaction mixture was stirred overnight at 50° C. After cooling, the purple solid was filtered and recrystallized from CHCl₃/Hex to give a pure product (0.42 g, 81%); mp. 180-182° C.

¹H NMR (CDCl₃, ppm): δ 1.15 (m, 6H) 1.97 (m, 2H), 2.95 (s, 3H), 3.45 (t, J=7.0 Hz, 2H), 3.88 (m, 4H), 4.22 (t, J=6.7 Hz, 2H), 6.61 (d, J=8.3 Hz, 2H), 6.67 (d, J=8.3 Hz, 2H), 6.84 (s, 2H), 7.26 (d, J=16 Hz, ═CH, 1H), 7.34 (d, J=16 Hz, ═CH, 1H), 7.47 (d, J=8.3 Hz, ArH, 2H), 7.73 (d, J=8.7 Hz, 2H), 8.10 (d, J=3.3 Hz, thienyl proton, 1H), 8.53 (s, 1H), 9.23 (s, OH, 2H). Anal. calcd for C₃₇H₃₆N₄O₅S: C, 68.50; H, 5.59; N, 8.64. Found: C, 68.24; H, 5.71; N, 8.65.

In a similar fashion, to a mixture of compound 9 in EtOH, was added compound 10b. The reaction mixture was stirred overnight at 50° C. After cooling, the purple solid was filtered and recrystallized from CHCl₃/Hex to give an 80% yield of a pure product; mp 178-180° C.

¹H NMR (CDCl₃, ppm): δ 1.27 (m, 6H), 2.13 (m, 2H), 2.91 (s, 3H), 3.37 (t, J=6.9 Hz, 2H), 4.17 (t, J=6.5 Hz, 2H), 4.59 (m, 4H), 6.48 (d, J=8.4 Hz, 2H), 6.73 (m, 3H), 6.84 (s, 2H), 7.06 (d, J=16.0 Hz, ═CH, 1H), 7.18 (d, J=4.1 Hz, thienyl proton, 1H), 7.30 (d, J=8.3 Hz, ArH, 2H), 7.34 (d, J=16.0 Hz, ═CH, 1H), 7.76 (d, J=8.5 Hz, ArH, 2H), 7.78 (d, J=3.8 Hz, thienyl proton, 1H), 8.45 (s, OH, 2H), 8.57 (s, 1H). Anal. calcd for C₃₇H₃₆N₄O₄S₂: C, 66.84; H, 5.46; N, 8.43. Found: C, 68.57; H, 5.60; N, 8.33.

In a similar fashion, to a mixture of compound 9 in EtOH, was added compound 10c. The reaction mixture was stirred overnight at 50° C. After cooling, the purple solid was filtered and recrystallized from CHCl₃/Hex to give a 40% yield of a pure product; mp 210-212° C.

¹H NMR (CDCl₃, ppm): δ 1.94 (m, 2H), 2.94 (s, 3H), 3.46 (t, J=7.0 Hz, 2H), 4.22 (t, J=7.0 Hz, 2H), 6.61 (d, J=8.0 Hz, 2H), 6.69 (d, J=8.0 Hz, 2H), 6.84 (s, 2H), 7.40 (s, HC═CH, 2H), 7.51 (d, J=9.0 Hz, ArH, 2H), 7.53 (d, J=4.0 Hz, thienyl proton, 1H), 7.72 (d, J=8.3 Hz, ArH, 2H), 8.04 (m, 3H), 8.21 (d, J=5.0 Hz, thienyl proton, 1H), 8.62 (d, J=7.0 Hz, 1H), 8.67 (s, 1H), 9.23 (s, OH, 2H). Anal. calcd for C₄₀H₃₀N₄O₄S₂: C, 69.14; H, 4.35; N, 8.06. Found: C, 69.28; H, 4.24; N, 8.03.

### Example 8

### Synthesis of Compound 14 from FIG. 2

To a stirred solution of 4-aminobenzoic acid 13 (1.41 g, 10.26 mmol) in 5 mL anhydrous NMP was added anhydride 12 (2.28 g, 5.1 mmol). The mixture was stirred under nitrogen at 90° C. for 12 hours, when the temperature was raised to 140° C. After being heated at 140° C. for 4 hours, the solution was cooled and poured into a 100 mL solution of H₂O/MeOH (7:3). The crude product was recrystalized from NMP/H₂O to give compound 14 as a white solid (2.65 g, 77%); mp 352-354° C.

¹H NMR (DMSO-d₆, ppm): δ 7.56 (d, J=8.5 Hz, 4H), 7.72 (s, ArH, 2H), 7.94 (d, J=8.0 Hz, ArH, 2H), 8.05 (d, J=8.5 Hz, ArH, 4H), 8.18 (d, J=8.0 Hz, ArH, 2H). C₃₃H₁₆F₆N₂O₈: C, 58.08; H, 2.36; N, 4.10. Found: C, 57.95; H, 2.43 N, 4.06.

### Example 9

### Synthesis of Polymers from Monomers 11a-c

Polymers were prepared from monomers 11a-c as follows. A solution of monomer 11a-c (0.30 mmol) and diacid 14 (0.30 mmol) and DPTS (1.20 mmol) in 2.5 mL anhydrous NMP under nitrogen, was treated dropwise with diisopropyl-carbodiimide (1.20 mmol) at 0° C. After the addition completed, the reaction mixture was stirred at room temperature for 24 hours. When the reaction was completed the solution was poured into MeOH (75 mL). The polymer was collected and redissolved in NMP (2-3 mL) then poured into MeOH (75 mL). The polymer was collected and washed with MeOH in Soxhlet extractor for 2 days then dried under vacuum at 50° C. for 24 hours.

For the polymer derived from monomer 11a: 92% yield.

¹H NMR (CDCl₃, ppm): δ 1.22 (s, NCH₂CH₃, 6H), 2.20 (s, NCH₂CH₂CH₂Ar, 2H), 2.95 (s, NCH₃, 3H), 3.41 (s, NCH₂CH₂CH₂Ar 2H), 4.01 (s, NCH₂CH₃, 4H), 4.30 (s, NCH₂CH₂CH₂Ar, 4H), 6.58 (s, ArH, 2H), 7.13 (s, ArH, 1H), 7.18 (s, ArH, 3H), 7.22 (s, ArH, 6H), 7.55 (s, ArH, 1H), 7.67 (s, ArH, 4H), 7.94(s, ArH, 4H), 8.08 (s, ArH, 4H), 8.41 (s, ArH, 3H), 8.50 (s, 1H). Anal. calcd for C₇₀H₄₈F₆N₆O₁₁S: C, 64.91; H, 3.74; N, 6.49. Found: C, 64.15; H, 3.89; N, 6.57.

For the polymer derived from monomer 11b: 91% yield.

¹H NMR (CDCl₃, ppm): δ 1.21 (s, NCH₂CH₃, 6H), 2.23 (s, NCH₂CH₂CH₂Ar, 2H), 2.70 (s, NCH₃, 3H), 3.42 (s, NCH₂CH₂CH₂Ar 2H), 4.23 (s, NCH₂CH₂CH₂Ar, 2H), 6.56 (s, ArH, 2H), 6.75 (s ArH,1H), 7.10 (s, ArH, 2H), 7.15 (s, ArH, 4H), 7.22 (s, ArH, 4H), 7.70 (s, ArH, 4H), 7.87 (s, ArH, 4H), 8.10 (s, ArH, 5H), 8.18 (s, ArH, 3H), 8.24 (s, ArH, 1H), 8.41 (s, AH, 2H), 8.48 (s, 1H). Anal. calcd for C₇₀H₄₈F₆N₆O₁₀S₂: C, 64.12; H, 3.69; N, 6.41. Found: C, 63.73; H, 4.04; N, 6.35.

For the polymer derived from monomer 11c: 92% yield.

¹H NMR (CDCl₃, ppm): δ 2.18 (s, NCH₂CH₂CH₂Ar, 2H), 2.92 (s, NCH₃, 3H), 3.82 (s, NCH₂CH₂CH₂Ar 2H), 4.35 (s, NCH₂CH₂CH₂Ar, 2H), 6.55 (s, ArH, 2H), 6.85 (s ArH, 1H), 7.07 (s, ArH, 2H), 7.17 (s, ArH, 3H), 7.25 (s, ArH, 3H), 7.54 (s, ArH, 4H), 7.75 (s, ArH, 7H), 7.95 (s, ArH, 5H), 8.08 (s, ArH, 1H), 8.34 (s, ArH, 3H), 8.45 (s, AH, 2H). Anal. calcd for C₇₃H₄₂F₆N₆O₁₀S₂: C, 64.91; H, 3.74; N, 6.49. Found: C, 64.15; H, 3.89; N, 6.57.

### Example 10

### Synthesis of Monomers 20a-c, as in FIG. 2, and Polymers from these Monomers Were Made in a Similar Fashion

### Example 11

### Physical Characterization

¹H NMR Spectra were recorded on a Bruker AM 400 spectrometer. UV-visible spectra were collected using a Shimadzu UV-2401 PC spectrophotometer. The GPC measurements were performed on a Waters RI system (available from Waters, Milford, Mass.) equipped with a UV detector and a differential refractometer detector using THF as an eluent. Molecular weight distributions were calculated based on monodispersed polystyrene standards. Thermal analyses were performed by using the DSC-10 and TGA-50 systems from TA instruments under a nitrogen atmosphere. The melting points were obtained with open capillary tubes on a MeI-Temp apparatus. Elemental analyses were performed by Atlantic Microlab, Inc, Norcross, Ga.

### Example 12

### Optical Measurements

The Teng and Man ellipsometric technique for the electro-optic coefficient measurements, as outlined in Teng, C. C.; Man; H. T. Appl. Phys. Lett. 1990, 56, 1734 was used. For these measurements, a cast polymer film on an Indium-Tin-Oxide (ITO) substrate was poled under a corona discharge at 170° C. While maintaining the corona discharge, the sample was cooled to room temperature. Silver electrodes with 0.1 micron thickness were evaporated on the polymer surface. The thickness and refractive index were measured by using a prizm-coupler, available from Metricon, Pennington, N.J.

The second harmonic generation (SHG) of the poled polymeric films was measured using a model-locked Nd:YAG laser (Continuum-PY61C-10 with a pulse width of 25 ps and a repetition rate of 10 Hz, available from Continuum, Santa Clara, Calif.) as a fundamental source (1.064 μm). A quartz crystal was used as the reference sample.

### Prophetic Example 1

### Synthesis of Crosslinkable Linking Monomers 34, 34-1, and 34-2.

One equivalent of compound 24 is reacted with 2 equivalents of compound 26 with Pd(PPh₃)₄/K₃PO₄ serving as a catalyst in dioxane for about 10 hours to give compound 28. Resultant compound 28 is then converted into compound 30 with NBS in dimethylsulfide. Reaction of compound 30 with TEMPO (Free radical) leads to the formation of compound 32, which is further treated with acid to prepare diphenol monomer 34. Crosslinkable linking monomer 34-2 is prepared in a similar fashion.

Other thermally sensitive radical precursors, in addition to TEMPO, can be also be used with the aromatic diphenol monomers, such as the AIBN derivative 34-1.

### Prophetic Example 2

### Coupling of Crosslinkable Linking Monomers with NLO Chromophoric Monomers 36a-d or 36-1a-d to Give Crosslinkable NLO Polymers 41a-d and 36a-d

Under mitsunobu reaction conditions (PPh₃/DEAD/DMF), monomer 34 and 36a-d can be polymerized to obtain crosslinkable NLO polyether 41a-d. Here, the polymerization is performed at low temperature, and the Mitsunobu reaction utilized. When other chromophoric monomers are substituted for ethanol amino monomers 36a-d, a similar polymerization strategy is applied. Crosslinkable linking monomers 34-2 and 34-1 (once acidified) are polymerized with chromophoric diol monomers using a similar strategy.

### Prophetic Example 3

### Synthesis of NLO Chromophoric Monomers 52a-c

Compound 42 is synthesized via silylation of corresponding diphenol and then reacted with compound 44 in a 1:1 ratio using an excess amount of NaH to obtain compound 46. Compound 46 is then reacted with 48 under Heck reaction conditions (5%, Pd(DBA/NBu/P(t-Bu)₃) to yield compound 50. The aldehyde group in compound 50 is then condensed with electron withdrawing groups Xa-c. The condensation product is then deprotected using NH₄F to generate diphenol chromophoric monomers 52a-c.

### Prophetic Example 4

### Synthesis of NLO Chromophores 60a-c and 66a-c

The coupling reaction between compounds 54 and 44 yields compound 56 which is then coupled with compound 48 under Heck reaction conditions (5%, Pd(DBA/NBu/P(t-Bu)₃). Condensation with electron withdrawing groups Xa-c generate a protected monohydroxy compound (not shown), which is then deprotected with mild NH₄F to yield NLO chromophores 60a-c. Chromophores 66a-c are similarly prepared.

### Prophetic Example 5

### Synthesis of Linking Monomers 68 and NLO Chromophoric Monomer 70

One equivalent of compound 68 is reacted with compounds two equivalents of 60a-c under Mitsunobu conditions (PPh₃/DEAD/DMF) to generate monomer 70. This monomer can polymerize with 2,5-di(tributylstananyl)thiophene under the Stille coupling conditions (Pd(PPh₃)₂Cl₂/PPh₃) to generate polyimides.

As any person of ordinary skill in the art of polymer chemistry will recognize from the provided description, figures, and examples, that modifications and changes can be made to the preferred embodiments of the invention without departing from the scope of the invention defined by the following claims and their equivalents.

What is claimed:

1. A compound, for forming a nonlinear optic material, comprising the structure:
X—Y—Z; wherein
X is 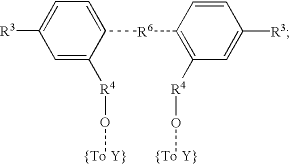
R³ comprises a labile group and is selected from the group consisting of halogens, halogen containing moieties, alcohols, alkoxy, oxygen containing moieties, carbonyl containing moieties, thiophene containing moieties, and mixtures thereof;
R⁴ is selected from the group consisting of a single bond and a carbonyl group;
R⁶ is an aromatic group comprising a carbonyl carbon and a nitrogen;
Y is 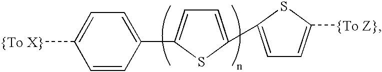
and n is an integer from 0 to 10; and Z is an electron withdrawing group,
wherein Y and Z in combination form a nonlinear optic chromophore.

2. The compound of claim 1, wherein said compound is substantially deuterated.

3. The compound of claim 1, wherein R³ is a halogen.

4. The compound of claim 1, wherein R⁶ is selected from the group consisting of 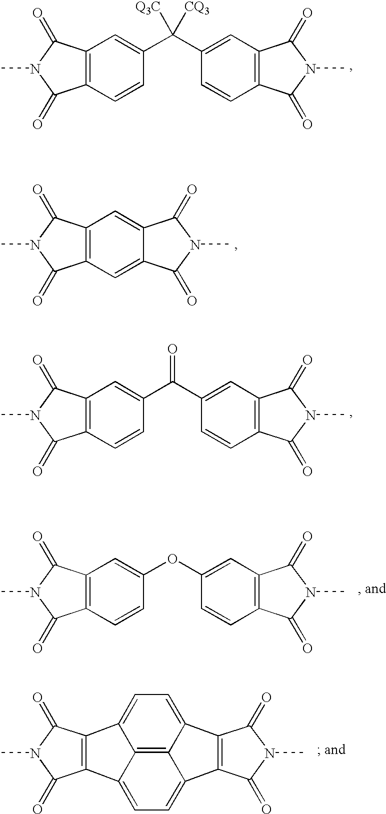
Q is a halogen.

5. The compound of claim 1, wherein said electron withdrawing group is selected from the group consisting of

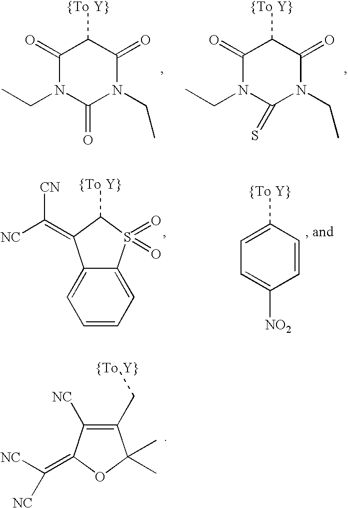

6. A compound, for forming a nonlinear optic material, comprising the structure:
X—Y—Z; wherein
X is 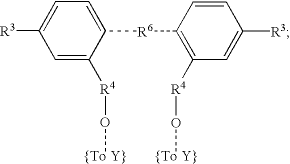
R³ comprises a labile group and is selected from the group consisting of halogens, halogen containing moieties, alcohols, alkoxy, oxygen containing moieties, carbonyl containing moieties, and thiophene containing moieties, or mixtures thereof;
R⁴ is selected from the group consisting of a single bond and a carbonyl group;
R⁶ is an aromatic group comprising a carbonyl carbon and a nitrogen;
Y is 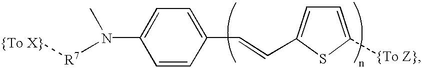
wherein
n is an integer from 1 to 10, and
R⁷ is selected from the group consisting of a saturated alkyl, an unsaturated alkyl, an aromatic, a heterosubstituted saturated alkyl, a heterosubstituted unsaturated alkyl, and a heterosubstituted aromatic; and
Z is 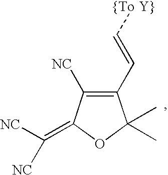
wherein Y and Z in combination form a nonlinear optic chromophore.

7. The compound of claim 6, wherein R⁷ is saturated alkyl.

8. The compound of claim 6, wherein R⁷ is C₃H₆.

9. A polymer comprising the compound of claim 1, wherein at least one said labile group is removed from said compound.

10. A polymer comprising the compound of claim 4, wherein at least one said labile group is removed from said compound.

11. A polymer comprising the compound of claim 5, wherein at least one said labile group is removed from said compound.

12. A polymer comprising the compound of claim 6, wherein at least one said labile group is removed from said compound.

13. The polymer of claim 9, further comprising a linking monomer.

14. The polymer of claim 13, wherein said linking monomer is selected from the group consisting of 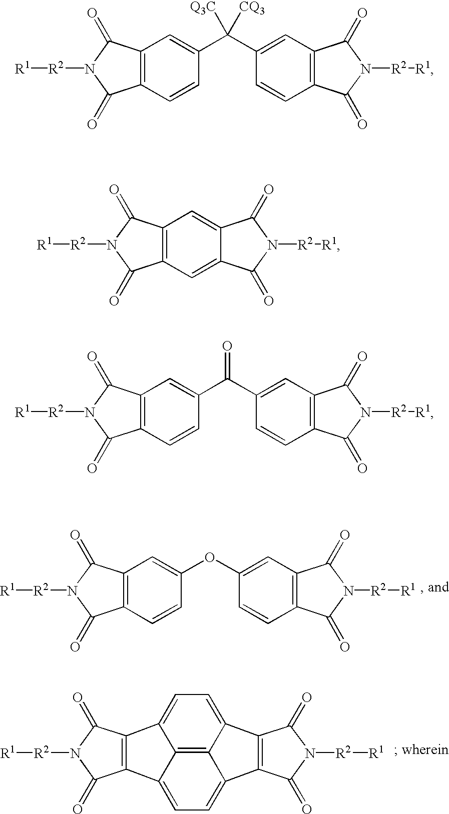
R¹ comprises a labile group;
R² is selected from the group consisting of single bond, carbonyl containing moiety, saturated alkyl, unsaturated alkyl, aromatic, substituted aromatic, heterosubstituted saturated alkyl, heterosubstituted unsaturated alkyl heterosubstituted aromatic, 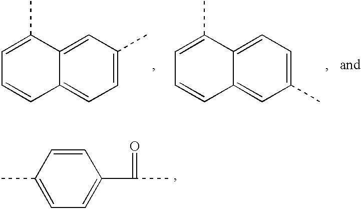
or mixtures thereof;
Q is a halogen; and
at least one said labile group is removed from said linking monomer.

15. The polymer of claim 9, further comprising at least two linking monomers, wherein at least one of said two linking monomers is a crosslinkable linking monomer comprising the structure 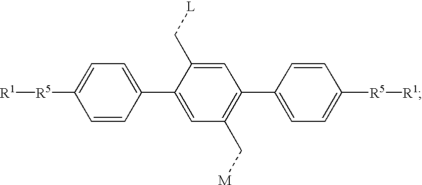
wherein
R¹ comprises a labile group;
R⁵ is selected from the group consisting of a single bond, an oxygen atom, a carbonyl group, a carbonyl containing moiety, and a thiophene containing moiety;
L is a crosslinking substituent;
M is L or a nonlinear optic chromophore; and
at least one said labile group is removed from said one linking monomer.

16. The polymer of claim 15, wherein said crosslinking substituent is selected from the group consisting of 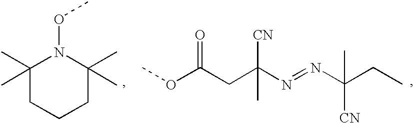
and mixtures thereof.

17. The polymer of claim 16, wherein a first crosslinking substituent and a second crosslinking substituent are crosslinked.

18. The polymer of claim 10, further comprising a linking monomer.

19. The polymer of claim 18, wherein said linking monomer is selected from the group consisting of 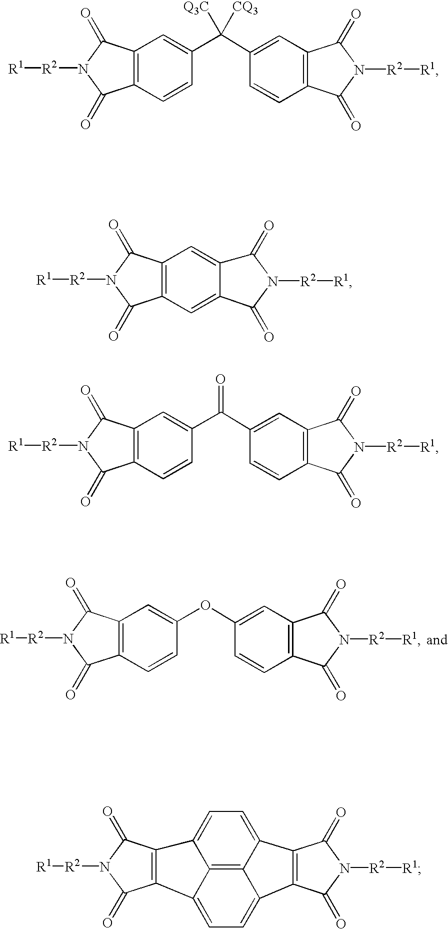
wherein
R¹ comprises a labile group;
R² is selected from the group consisting of single bond, carbonyl containing moiety, saturated alkyl, unsaturated alkyl, aromatic, substituted aromatic, heterosubstituted saturated alkyl, heterosubstituted unsaturated alkyl, heterosubstituted aromatic, 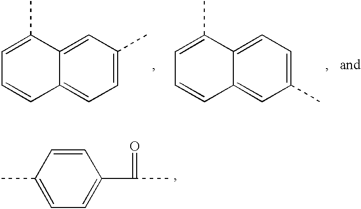
or mixtures thereof;
Q is a halogen; and
at least one said labile group is removed from said linking monomer.

20. The polymer of claim 10, further comprising at least two linking monomers, wherein at least one of said two linking monomers is a crosslinkable linking monomer comprising the structure 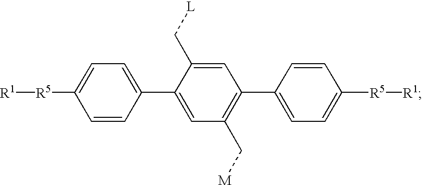
wherein
R¹ comprises a labile group;
R⁵ is selected from the group consisting of a single bond, an oxygen atom, a carbonyl group, a carbonyl containing moiety, and a thiophene containing moiety;
L is a crosslinking substituent;
M is L or a nonlinear optic chromophore; and
at least one said labile group is removed from said one linking monomer.

21. The polymer of claim 20, wherein said crosslinking substituent is selected from the group consisting of 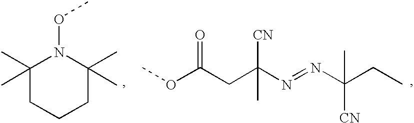
and mixtures thereof.

22. The polymer of claim 21, wherein a first crosslinking substituent and a second crosslinking substituent are crosslinked.

23. The polymer of claim 11, further comprising a linking monomer.

24. The polymer of claim 23, wherein said linking monomer is selected from the group consisting of 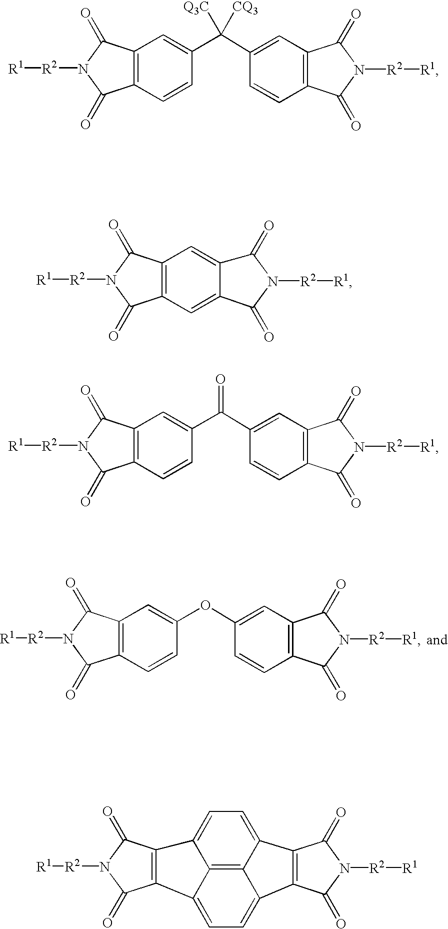
wherein
R¹ comprises a labile group;
R² is selected from the group consisting of single bond, carbonyl containing moiety, saturated alkyl, unsaturated alkyl, aromatic, substituted aromatic, heterosubstituted saturated alkyl, heterosubstituted unsaturated alkyl, heterosubstituted aromatic, 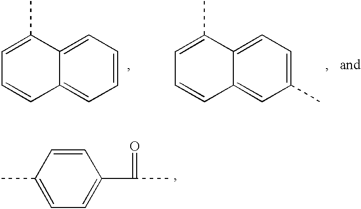
or mixtures thereof;
Q is a halogen; and
at least one said labile group is removed from said linking monomer.

25. The polymer of claim 11, further comprising at least two linking monomers, wherein at least one of said two linking monomers is a crosslinkable linking monomer comprising the structure 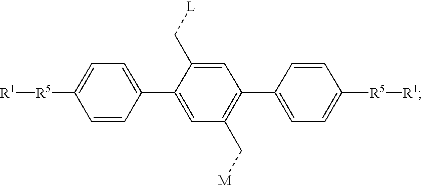
wherein
R¹ comprises a labile group;
R⁵ is selected from the group consisting of a single bond, an oxygen atom, a carbonyl group, a carbonyl containing moiety, and a thiophene containing moiety;
L is a crosslinking substituent;
M is L or a nonlinear optic chromophore; and
at least one said labile group is removed from said one linking monomer.

26. The polymer of claim 25, wherein said crosslinking substituent is selected from the group consisting of 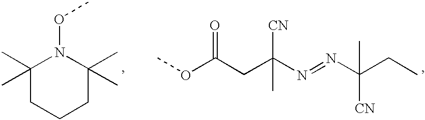
and mixtures thereof.

27. The polymer of claim 26, wherein a first crosslinking substituent and a second crosslinking substituent are crosslinked.

28. The polymer of claim 12, further comprising a linking monomer.

29. The polymer of claim 28, wherein said linking monomer is selected from the group consisting of 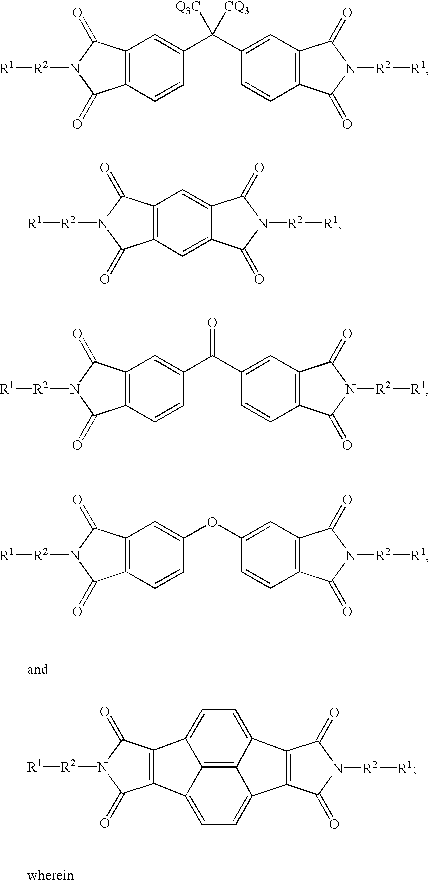
wherein
R¹ comprises a labile group;
R² is selected from the group consisting of single bond, carbonyl containing moiety, saturated alkyl, unsaturated alkyl, aromatic, substituted aromatic, heterosubstituted saturated alkyl, heterosubstituted unsaturated alkyl, heterosubstituted aromatic, 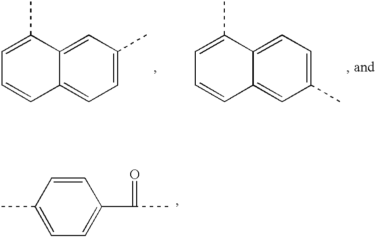
or mixtures thereof;
Q is a halogen; and
at least one said labile group is removed from said linking monomer.

30. The polymer of claim 12, further comprising at least two linking monomers, wherein at least one of said two linking monomers is a crosslinkable linking monomer comprising the structure 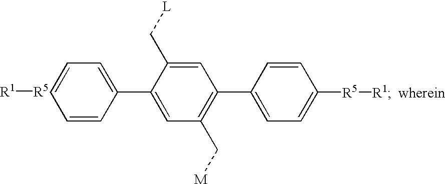
wherein
R¹ comprises a labile group;
R⁵ is selected from the group consisting of a single bond, an oxygen atom, a carbonyl group, a carbonyl containing moiety, and a thiophene containing moiety;
L is a crosslinking substituent;
M is L or a nonlinear optic chromophore; and
at least one said labile group is removed from said one linking monomer.

31. The polymer of claim 30, wherein said crosslinking substituent is selected from the group consisting of 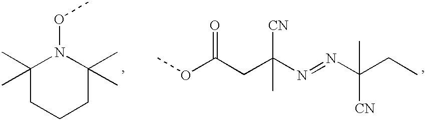
and mixtures thereof.

32. The polymer of claim 31, wherein a first crosslinking substituent and a second crosslinking substituent are crosslinked.

33. Polymerizing a composition comprising the compound of claim 1.

34. Polymerizing a composition comprising the compound of claim 4.

35. Polymerizing a composition comprising the compound of claim 5.

36. Polymerizing a composition comprising the compound of claim 6.

37. Polymerizing a composition comprising the compound of claim 8.

38. An electro-optical device comprising the polymer of claim 9, 10,11, 13,14,15,17,18, 19, 20, 22, 23, 24, 25, 27, 28, 29, 30, or 32, wherein said device has an electro-optically variable refractive index.

39. The electro-optical device of claim 38, wherein said device is selected from the group consisting of a phase modulator, a light intensity modulator, a directional coupler, an optical switch, an optical waveguide, and bulk devices having variable indices of refraction.
